(19) United States
(12) Patent Application Publication
(10) Pub. No.: US 2020/0376121 A1
(43) Pub. Date: Dec. 3, 2020

(54) BIOPHOTONIC COMPOSITIONS, METHODS, AND KITS FOR PAIN RELIEF

(71) Applicant: KLOX TECHNOLOGIES INC., Laval (CA)

(72) Inventors: Andreas NIKOLIS, Ville Mont-Royal (CA); Marco ROMANELLI, Pisa (IT); Nikolaos LOUPIS, Athens (GR); Remigio PIERGALLINI, San Benedetto del Tronto (IT); Valentina DINI, Pisa (IT)

(21) Appl. No.: 16/333,794

(22) PCT Filed: Sep. 22, 2017
(86) PCT No.: PCT/CA2017/051122

§ 371 Date: Mar. 15, 2019

Related U.S. Application Data
(60) Provisional application No. 62/399,192, filed on Sep. 23, 2016.

(51) Int. Cl. A61K 41/00; A61P 17/02; A61K 31/728; A61K 31/7008; A61K 31/4166

(52) U.S. Cl. CPC A61K 41/008; A61P 17/02; A61K 31/4166; A61K 31/7008; A61K 31/728

(57) ABSTRACT

The present document describes methods and uses of compositions which comprise at least one photoactivator or chromophore in association with a pharmacologically acceptable carrier for use in reducing pain that is associated with a medical condition in a subject.

### TECHNICAL FIELD

This description relates to the field of compositions, methods, and uses for pain reduction or relief.

### BACKGROUND OF THE INVENTION

Pain is a complex sensation which is mediated by the nervous system and is perceived as an unpleasant sensory and emotional experience. The highly personal and subjective perception of pain makes it difficult to define and treat clinically. The way in which each person perceives pain and its severity depends on numerous factors such as the type of illness, injury, and other biological and environmental factors.

Traditional treatment of pain uses analgesic drugs which act in various ways on the peripheral and central nervous systems to relieve pain. An analgesic drug refers to a drug whose chemical structure and physiological effects are such that when the drug is administered, it then acts to relieve pain. To qualify as an analgesic, the drug must reduce or abolish pain without, however, impairing consciousness, causing mental confusion or otherwise deranging the nervous system. Thus a drug which renders the patient unconscious is not an analgesic, but an anesthetic, even though it abolishes pain.

Narcotic alkaloids are a well-known class of analgesics. The oldest and best known are opium and morphine, its most active alkaloid. Also derived from opium is codeine. A person, who takes opium or its derivatives for a prolonged period, needs and tolerates larger and larger doses to obtain the desired effect, and therefore, becomes habituated. Should the use of the drug be then stopped, highly disagreeable withdrawal symptoms are experienced. Narcotic alkaloids are all potentially addictive, and an addict will often go to great lengths to obtain the narcotic to avoid withdrawal symptoms. Among non-narcotic analgesic drugs are the salicylates, such as aspirin. But these are far less effective in reducing pain than morphine and synthetic opioids.

Reducing the pain associated with medical conditions, such as for example relating to skin or soft tissues, is a complex and challenging endeavor. For instance, wounds frequently have non-resistant or resistant infections which lead to pain in the subject. Treatment of the pain associated with these wounds would be beneficial in mammals such as humans, horses, cats, or dogs. Thus an effective method for reducing and relieving pain, such as pain associated with skin and soft tissue wounds having non-resistant infections or resistant infections or both categories of infections, is needed.

### SUMMARY OF THE INVENTION

The biophotonic therapy of the disclosure provides new methods of reducing or relieving pain, such as pain associated with a medical condition, e.g., a medical condition related to the skin or soft tissues of a subject. The compositions, methods, kits, and uses disclosed herein reduce pain in a non-systemic manner as compared to conventional treatments, thereby resulting in less stress and discomfort to the patient as well as avoiding complications of administering different medications at the same time. The compositions, methods, and kits disclosed herein are also easy and convenient to use. A subject in need can apply any composition as described in this disclosure directly to the area of pain. The compositions, methods, and kits of the disclosure can be used to reduce or relieve pain, such as chronic or acute pain. In certain embodiments, the compositions, methods, and kits of the disclosure can be used in reducing nociceptive or neuropathic pain.

In some aspects, this disclosure provides a method of reducing pain associated with a medical condition in a subject, comprising topically applying a composition comprising at least one photoactivator and a pharmaceutically acceptable carrier, and exposing said composition to actinic light to cause activation of the composition, wherein the pain associated with the medical condition is treated non-systemically. In certain such aspects, the composition is a biophotonic composition. In certain such aspects, the composition is a topical biophotonic composition.

In certain aspects, the pain does not originate from a stimulus in an orofacial region. In certain such aspects, the reduction of pain is not dependent on the treatment of the underlying medical condition(s). In certain aspects, the medical condition is associated with the skin or soft tissues. In certain aspects, the pain is acute or chronic pain. In certain aspects, the pain is associated with post-surgical wounds. In certain aspects, the subject is a mammal, such as a human, an equine, a feline, or a canine.

In some embodiments of any of the foregoing or following, the photoactivator or chromophore of the composition is chosen from a xanthene derivative dye, an azo dye, a biological stain, and a carotenoid. In certain such embodiments, said xanthene derivative dye is chosen from a fluorene dye (e.g., a pyronine dye, such as pyronine Y or pyronine B, or a rhodamine dye, such as rhodamine B, rhodamine G, or rhodamine WT), a fluorone dye (e.g., fluorescein, or fluorescein derivatives, such as phloxine B, rose bengal, merbromine, Eosin Y, Eosin B, or Erythrosine B, i.e., Eosin Y), or a rhodole dye. In certain such embodiments, said azo dye is chosen from methyl violet, neutral red, para red, amaranth, carmoisine, allura red AC, tartrazine, orange G, ponceau 4R, methyl red, and murexide-ammonium purpurate. In certain such embodiments, said biological stain is chosen from safranin O, basic fuchsin, acid fuschin, 3,3′ dihexylocarbocyanine iodide, carminic acid, and indocyanine green. In certain such embodiments, said carotenoid is chosen from crocetin, a-crocin (S,S-diapo-S,S-carotenoic acid), zeaxanthine, lycopene, α-carotene, β-carotene, bixin, and fucoxanthine. In certain such embodiments, said carotenoid is present in the composition as a mixture chosen from saffron red powder, annatto extract, and brown algae extract.

In some embodiments of any of the foregoing or following, the composition further comprises at least one oxidant. In certain such embodiments, the oxidant is chosen from hydrogen peroxide, carbamide peroxide and benzoyl peroxide. In other embodiments, the oxidant is chosen from a peroxy acid and an alkali metal percarbonate.

In some embodiments of any of the foregoing or following, this disclosure provides a method of reducing pain associated with a medical condition in a subject, comprising topically applying a composition comprising at least one oxidant and at least one photoactivator or chromophore capable of activating the at least one oxidant; and exposing said composition to actinic light to cause activation of the composition (e.g., to cause activation of the at least one oxidant of the composition), wherein the pain associated with the medical condition is treated non-systemically. In certain embodiments, the composition is a biophotonic composition. In certain such embodiments, the composition is a topical biophotonic composition. In certain such embodiments, the pain does not originate from a stimulus in an orofacial region. In certain such embodiments, the reduction of pain is not dependent on the treatment of the underlying medical condition(s).

In certain aspects of the disclosure, said composition does not comprise an oxidant selected from the group consisting of a peroxide, a peroxy acid, hydrogen peroxide, carbamide peroxide, an alkali metal peroxide, an alkali metal percarbonate, peroxyacetic acid, and an alkali metal perborate. In certain such aspects, the undesired side effects caused by such oxidants may be reduced, minimized, or prevented.

In certain embodiments of any of the foregoing or following, the composition further comprises one or more salts selected from the group consisting of one or more bicarbonate salts, one or more carbonate salts, and a combination of the foregoing salts.

In certain aspects, the disclosure provides a method of reducing pain associated with a medical condition in a subject, comprising topically applying a composition comprising at least one photoactivator; at least one salt selected from the group consisting of one or more bicarbonate salts, one or more carbonate salts, and a combination of the foregoing salts; and a pharmaceutically acceptable carrier; and exposing said composition to actinic light to cause activation of the composition, wherein the pain associated with the medical condition is treated non-systemically. In certain aspects, the composition is a biophotonic composition. In certain such aspects, the composition is a topical biophotonic composition.

In some embodiments of any of the foregoing or following, the composition further comprises at least one healing factor chosen from hyaluronic acid, glucosamine, and allantoin.

In certain embodiments of any of the foregoing or following, said composition further comprises at least one chelating agent chosen from ethylenediaminetetraacetic acid (EDTA) and ethylene glycol tetraacetic acid (EGTA). In certain embodiments of any of the foregoing or following, the chelating agent is EDTA.

In some embodiments of any of the foregoing or following, the composition further comprises at least one gelling agent, such as glucose, modified starch, methyl cellulose, carboxymethyl cellulose, propyl cellulose, hydroxypropyl cellulose, a carbomer, alginic acid, sodium alginate, potassium alginate, ammonium alginate, calcium alginate, agar, carrageenan, locust bean gum, pectin, or gelatin.

In some embodiments of any of the foregoing or following, a composition of the disclosure is exposed to actinic light for a period of less than about 9 minutes, e.g., for a period of from about 1 second to about 8 minutes, from about 1 minute to about 8 minutes, from about 2 minutes to about 7 minutes, from about 3 minutes to about 6 minutes, from about 4 minutes to about 5 minutes. In certain embodiments, said composition is exposed to actinic light for a period of less than about 5 minutes per cm² of an area to be treated, e.g., for a period of about 1 second to about 5 minutes per cm². In certain embodiments, said composition is exposed to actinic light for a period of about 5 minutes per cm² of an area to be treated.

In some embodiments of any of the foregoing or following, the source of actinic light is placed over an area to be treated. In some embodiments, said actinic light is visible light having a wavelength between about 400 nm and about 700 nm. In some embodiments, said actinic light is illuminating in continuous motion over an area to be treated.

In some aspects, the disclosure provides for use of a composition for the manufacture of a medicament for reducing pain associated with a medical condition in a subject, wherein said composition comprises at least one photoactivator and a pharmaceutically acceptable carrier; wherein the pain associated with the medical condition is treated non-systemically. In some aspects, the composition further comprises at least one oxidant. In certain such aspects, the photoactivator is capable of activating the oxidant. In other aspects, the composition further comprises one or more salts selected from the group consisting of one or more bicarbonate salts, one or more carbonate salts, and a combination of the foregoing salts.

In some aspects, the disclosure provides for use of a composition for reducing pain that is associated with a medical condition in a subject, the composition comprising: at least one photoactivator and a pharmaceutically acceptable carrier, and wherein the pain associated with the medical condition is treated non-systemically. In certain aspects, the composition further comprises at least one oxidant. In certain such aspects, the photoactivator is capable of activating the oxidant. In other aspects, the composition further comprises one or more salts selected from the group consisting of one or more bicarbonate salts, one or more carbonate salts, and a combination of the foregoing salts.

### Definitions

Before continuing to describe the present disclosure in further detail, it is to be understood that this disclosure is not limited to specific compositions or process steps, as such may vary. It must be noted that, as used in this specification and the appended embodiments, the singular form “a”, “an” and “the” include plural referents unless the context clearly dictates otherwise.

As used herein, the term “about” in the context of a given value or range refers to a value or range that is within 20%, within 10%, and more within 5% of the given value or range.

It is convenient to point out here that “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

The term “reducing pain” or “relieving pain” as used herein refers to a reduction of the sensation of pain by the subject. In certain embodiments, pain reduction is characterized by a decrease in the visual analogue scale (VAS) score by at least 1 point or a reduction in the pain as scored by the physician's assessment methods.

The term “stimulus” as used herein refers to any event which results in the generation of action potential along the nerve. Event as used herein includes pain that is the result of an external stimulus such as a wound (acute or chronic) as well as pain that result from medical conditions such as arthritic pain or neuropathic pain that is the result of prior tissue injury.

The term “biophotonic” as used herein refers to the generation, manipulation, detection and application of photons in a biologically relevant context. In other words, compositions exert their physiological effects primarily due to the generation and manipulation of photons.

The term “Composition” is a composition as described herein that may be activated by light to produce photons for biologically relevant applications.

The term “orofacial” as used herein refers to any region pertaining to the mouth and face.

The term “healing factor” as used herein refers to a compound that promotes or enhances the healing or regenerative process of a tissue.

The term “acute pain” as used herein refers to any pain lasting less than 12 weeks.

The term “chronic pain” as used herein refers to any pain lasting more than 12 weeks.

The term “topical” means as applied to body surfaces, such as the skin, mucous membranes, vagina, oral cavity, internal surgical wound sites, and the like.

Terms “photoactivator”, “chromophore”, and “photoactivating agent” are used herein interchangeably. A chromophore refers to a compound, when contacted by light irradiation, is capable of absorbing the light. The chromophore readily undergoes photoexcitation and can then transfer its energy to other molecules or emit it as light.

The term “oxidant” as used herein refers to either a compound that readily transfers oxygen atoms and oxidizes other compounds, or a substance that gains electrons in a redox chemical reaction.

The term “chelating agent” as used herein refers to a compound that binds metal ions, such as iron, and facilitates their solvation in solution.

The term “gels” as used herein refers to substantially dilute cross-linked systems. Gels may be semi-solids and exhibit substantially no flow when in the steady state at room temperature (e.g. about 20-25° C.). By steady state is meant herein during a treatment time and under treatment conditions. Gels, as defined herein, may be physically or chemically cross-linked. As defined herein, gels also include gel-like compositions such as viscous liquids.

The term “oxidant” as used herein refers to either a compound that readily transfers oxygen atoms and oxidizes other compounds, or a substance that gains electrons in a redox chemical reaction.

The term “chelating agent” as used herein refers to a compound that binds metal ions, such as iron, and facilitates their solvation in solution.

The term “healing factor” as used herein refers to a compound that promotes or enhances the healing or regenerative process of a tissue.

The term “active oxygen species” as used herein refers to chemically-reactive molecules containing oxygen. Examples include oxygen ions and peroxides. They can be either inorganic or organic. Active oxygen species are highly reactive due to the presence of unpaired valence shell electrons. They are also referred to as “reactive oxygen”, “active oxygen”, or “reactive oxygen species”.

The “initial level of fluorescence” is the level of fluorescence exhibited by a composition of the disclosure immediately upon application of or activation with light.

The term “photobleaching” as used herein refers to the photochemical destruction of a chromophore.

The term “actinic light” as used herein refers to light energy emitted from a specific light source (e.g., lamp, LED, or laser) and capable of being absorbed by matter (e.g. the chromophore or photoactivator defined above). In some embodiments, the actinic light is visible light.

Features and advantages of the subject matter hereof will become more apparent in light of the following detailed description of selected embodiments, as illustrated in the accompanying figures. As will be realized, the subject matter disclosed is capable of modifications in various respects, all without departing from the scope of the disclosed embodiments. Accordingly, the figures and the description are to be regarded as illustrative in nature, and not as restrictive and the full scope of the subject matter is set forth in the disclosed embodiments.

### BRIEF DESCRIPTION OF THE DRAWINGS

Further features and advantages of the present disclosure will become apparent from the following detailed description, taken in combination with the appended drawings, in which:

FIG. 1 illustrates the Stokes' shift.

FIG. 2 illustrates the absorption and emission spectra of donor and acceptor chromophores. The spectral overlap between the absorption spectrum of the acceptor chromophore and the emission spectrum of the donor chromophore is also shown.

FIG. 3 is a schematic of a Jablonski diagram that illustrates the coupled transitions involved between a donor emission and acceptor absorbance.

FIG. 4 shows the VAS assessment of pain associated with wounds. The results indicated that for all ten patients experienced a reduction in their pain scores by the time of having received three treatments with the biophotonic composition (at T2), with seven of the ten patients reporting a complete absence of pain by the T2 point.

FIG. 5 shows a mean change from baseline, with respect to a reduction of pain experienced by the patients receiving the biophotonic composition treatment in comparison with those receiving the Silicone Sheets treatment following their breast reduction surgery.

FIG. 6 shows the relative wound area regression for venous leg ulcers, diabetic foot ulcers, and pressure ulcers treated with the biophotonic composition during a period of 20 weeks.

FIG. 7 shows the amount of redness, pain, heat, pus, and swelling in wounds and peri-wound skin at the first treatment visit, week 4, and week 12.

### DETAILED DESCRIPTION

In one aspect, the disclosure provides a method of reducing pain associated with a medical condition in a subject, comprising: a) topically applying a composition comprising at least one photoactivator and a pharmaceutically acceptable carrier; and b) exposing said composition to actinic light to cause activation of the composition, wherein the pain associated with the medical condition is treated non-systemically. In certain such embodiments, the composition is a biophotonic composition. In certain such embodiments, the composition is a biophotonic topical composition. In certain such embodiments, the pain does not originate from a stimulus in an orofacial region. In certain such embodiments, the reduction of pain is not dependent on the treatment of the underlying medical condition.

In certain aspects, the disclosure provides a method of treating or reducing pain in patients suffering from venous leg ulcer (VLU), comprising: a) topically applying a composition comprising at least one photoactivator and a pharmaceutically acceptable carrier; and b) exposing said composition to actinic light to cause activation of the composition, wherein the pain associated with VLU is treated non-systemically. In certain such embodiments, the composition is a biophotonic composition. In certain such embodiments, the composition is a biophotonic topical composition. In certain such embodiments, the pain does not originate from a stimulus in an orofacial region. In certain such embodiments, the reduction of pain is not dependent on the treatment of the underlying medical condition.

In certain aspects, the disclosure provides a method of treating or reducing pain in patients suffering from diabetic foot ulcer (DFU), comprising: a) topically applying a composition comprising at least one photoactivator and a pharmaceutically acceptable carrier; and b) exposing said composition to actinic light to cause activation of the composition, wherein the pain associated with DFU is treated non-systemically. In certain such embodiments, the composition is a biophotonic composition. In certain such embodiments, the composition is a biophotonic topical composition. In certain such embodiments, the pain does not originate from a stimulus in an orofacial region. In certain such embodiments, the reduction of pain is not dependent on the treatment of the underlying medical condition.

In certain aspects, the disclosure provides a method of treating or reducing pain in patients suffering from pressure ulcer (PU), comprising: a) topically applying a composition comprising at least one photoactivator and a pharmaceutically acceptable carrier; and b) exposing said composition to actinic light to cause activation of the composition, wherein the pain associated with PU is treated non-systemically. In certain such embodiments, the composition is a biophotonic composition. In certain such embodiments, the composition is a biophotonic topical composition. In certain such embodiments, the pain does not originate from a stimulus in an orofacial region. In certain such embodiments, the reduction of pain is not dependent on the treatment of the underlying medical condition.

In certain embodiments of any of the foregoing or following, the photoactivator is selected from the group consisting of a xanthene derivative dye, an azo dye, a biological stain, and a carotenoid. In certain such embodiments, the at least one photoactivator is selected from the group consisting of eosin (e.g., eosin B or eosin Y), erythrosine (e.g., erythrosine B), and Saffron red powder.

In certain embodiments of any of the foregoing or following, the composition comprises at least one photoactivator present in an amount of at least about 0.2% by weight of the composition, e.g., from about 0.02% to about 12%, from about 0.02% to about 10%, from about 0.02% to about 8%, from about 0.02% to about 6%, from about 0.02% to about 4%, from about 0.02% to about 2%, from about 0.02% to about 1%, from about 0.02% to about 2%, or about 0.5% by weight of the composition.

In some embodiments of any of the foregoing or following, the composition further comprises an additional photoactivator, such as a photoactivator selected from the group consisting of Xanthene derivative dye, azo dye, biological stain, and carotenoid.

In some embodiments of any of the foregoing or following, the chromophore or photoactivator of the composition is selected from the group consisting of a xanthene derivative dye, an azo dye, a biological stain, and a carotenoid. In certain such embodiments, said xanthene derivative dye is chosen from a fluorene dye (e.g., a pyronine dye, such as pyronine Y or pyronine B, or a rhodamine dye, such as rhodamine B, rhodamine G, or rhodamine WT), a fluorone dye (e.g., fluorescein, or fluorescein derivatives, such as phloxine B, rose bengal, merbromine, Eosin Y, Eosin B, or Erythrosine B, i.e., Eosin Y), or a rhodole dye. In certain such embodiments, said azo dye is chosen from methyl violet, neutral red, para red, amaranth, carmoisine, allura red AC, tartrazine, orange G, ponceau 4R, methyl red, and murexide-ammonium purpurate. In certain such embodiments, said biological stain is chosen from safranin O, basic fuchsin, acid fuschin, 3,3′ dihexylocarbocyanine iodide, carminic acid, and indocyanine green. In certain such embodiments, said carotenoid is chosen from crocetin, a-crocin (S,S-diapo-S,S-carotenoic acid), zeaxanthine, lycopene, α-carotene, β-carotene, bixin, and fucoxanthine. In certain such embodiments, said carotenoid is present in the composition as a mixture is selected from the group consisting of saffron red powder, annatto extract, and brown algae extract.

In some embodiments of any of the foregoing or following, the additional photoactivator is selected from a group consisting of phloxine B, rose bengal, eosin B, fluorescein, erythrosine B, rhodamine B, rhodamine G, rhodamine WT, saffron red powder, annatto extract, brown algae extract, safranin O, basic fuchsin, acid fuschin, 3,3′ dihexylocarbocyanine iodide, carminic acid, indocyanine green, crocetin, α-crocin (8,8-diapo-8,8-carotenoic acid), zeaxanthine, lycopene, α-carotene, β-carotene, bixin, fucoxanthine, methyl violet, neutral red, para red, amaranth, carmoisine, allura red AC, tartrazine, orange G, ponceau 4R, methyl red, murexide-ammonium purpurate, pyronine Y and pyronine B.

In certain embodiments of any of the foregoing or following, the additional photoactivator is present in an amount of from about 0.02% to about 12% by weight of the composition, such as from about 0.02% to about 10%, from about 0.02% to about 8%, from about 0.02% to about 6%, from about 0.02% to about 4%, from about 0.02% to about 2%, from about 0.02% to about 1%, from about 0.02% to about 2%, or about 0.5% by weight of the composition.

In some embodiments of any of the foregoing or following, the composition further comprises at least one oxidant. In certain such embodiments, the oxidant is selected from the group consisting of hydrogen peroxide, carbamide peroxide and benzoyl peroxide. In other embodiments, the oxidant is a peroxy acid or an alkali metal percarbonate.

In some embodiments of any of the foregoing or following, the photoactivator or chromophore is capable of activating the oxidant.

In some embodiments of any of the foregoing or following, the oxidant is present in an amount of from about 1% to about 70% by weight of the composition, e.g., from about 1% to about 60%, from about 1% to about 50%, from about 1% to about 40%, from about 1% to about 30%, from about 1% to about 20%; from about 1% to about 16%, from about 1% to about 14%, from about 1% to about 12%, from about 1% to about 10%, from about 1% to about 8%, from about 1% to about 6%, from about 0.05% to about 6%, from about 0.1% to about 6%, from about 0.5% to about 6%, from about 2.5% to about 6%, from about 3.5% to about 6% by weight of the composition.

In some embodiments of any of the foregoing or following, the oxidant is hydrogen peroxide and is present in an amount of from about 3.5% to about 6% by weight of the composition.

In some embodiments of any of the foregoing or following, the oxidant is carbamide peroxide and is present in an amount of from about 10% to about 16% by weight of the composition.

In some embodiments of any of the foregoing or following, the oxidant is benzoyl peroxide and is present in an amount of from about 2.5% to about 5% by weight of the composition.

In some embodiments of any of the foregoing or following, this disclosure provides a method of reducing pain associated with a medical condition in a subject, comprising topically applying a composition comprising at least one oxidant and at least one photoactivator, such as a photoactivator capable of activating the oxidant; and exposing said composition to actinic light to cause activation of the composition (e.g., to cause activation of the oxidant), wherein the pain associated with the medical condition is treated non-systemically. In certain such embodiments, the composition is a biophotonic composition. In certain such embodiments, the pain does not originate from a stimulus in an orofacial region. In certain such embodiments, the reduction of pain is not dependent on the treatment of the underlying medical condition.

In some embodiments of any of the foregoing or following, said composition does not comprise an oxidant selected from the group consisting of a peroxide, a peroxy acid, hydrogen peroxide, carbamide peroxide, an alkali metal peroxide, an alkali metal percarbonate, peroxyacetic acid, and an alkali metal perborate. In certain such embodiments, the undesired side effects caused by such oxidants may be reduced, minimized, or prevented.

In some embodiments of any of the foregoing or following, the composition further comprises at least one salt selected from the group consisting of one or more bicarbonate salts, one or more carbonate salts, and a combination of the foregoing salts.

In some embodiments of any of the foregoing or following, the at least one bicarbonate salt is selected from the group consisting of ammonium bicarbonate, caesium bicarbonate, potassium bicarbonate, sodium bicarbonate, choline bicarbonate, aminoguanidine bicarbonate, and tetraethylammonium bicarbonate. In certain such embodiments, the bicarbonate salt is sodium bicarbonate or potassium bicarbonate.

In some embodiments of any of the foregoing or following, the at least one carbonate salt is selected from the group consisting of barium carbonate, beryllium carbonate, caesium carbonate, calcium carbonate, cobalt (II) carbonate, copper (II) carbonate, lithium carbonate, magnesium carbonate, nickel (II) carbonate, potassium carbonate, sodium carbonate, and zinc carbonate. In certain such embodiments, the carbonate salt is selected from the group consisting of sodium carbonate, calcium carbonate, and potassium bicarbonate

In some embodiments of any of the foregoing or following, the composition further comprises at least one healing factor, such as hyaluronic acid, glucosamine, or allantoin.

In certain embodiments of any of the foregoing or following, said composition further comprises at least one chelating agent chosen from ethylenediaminetetraacetic acid (EDTA) and ethylene glycol tetraacetic acid (EGTA). In certain embodiments of any of the foregoing or following, the chelating agent is EDTA.

In certain embodiments of any of the foregoing or following, the composition further comprises at least one hydrophilic gelling agent. In certain such embodiments, the hydrophilic gelling agent is selected from the group consisting of glucose, modified starch, methyl cellulose, carboxymethyl cellulose, propyl cellulose, hydroxypropyl cellulose, Carbopol® polymers, alginic acid, sodium alginate, potassium alginate, ammonium alginate, calcium alginate, agar, carrageenan, locust bean gum, pectin, and gelatin. The hydrophilic gelling agent enhances the consistency of the composition and contributes to facilitating the application of the composition to the skin or area of biofilms.

In some embodiments of any of the foregoing or following, the disclosure provides a method for reducing pain associated with a medical condition in a subject, comprising topically applying a composition comprising at least one photoactivator; at least one salt selected from the group consisting of one or more bicarbonate salts, one or more carbonate salts, and a combination of the foregoing salts; and a pharmaceutically acceptable carrier; and exposing said composition to actinic light to cause activation of the composition, wherein the pain associated with the medical condition is treated non-systemically. In certain such embodiments, the composition is a biophotonic composition. In certain such embodiments, the pain does not originate from a stimulus in an orofacial region. In certain such embodiments, the reduction of pain is not dependent on the treatment of the underlying medical condition.

In some embodiments of any of the foregoing or following, said composition is exposed to actinic light, such as a LED light, for at least one treatment period of from about 1 minute to about 30 minutes per cm² of an area to be treated. In certain such embodiments, said composition is exposed to actinic light for at least one treatment period of from about 4 minutes to about 26 minutes, from about 8 minutes to about 24 minutes, from about 10 minutes to about 20 minutes, from about 10 minutes to about 18 minutes; or from about 1 minutes to about 5 minutes, from about 5 minutes to about 10 minutes, from about 10 minutes to about 15 minutes, from about 15 minutes to about 20 minutes, from about 20 minutes to about 25 minutes, or about 5 minutes, about 10 minutes, about 15 minutes, or about 20 minutes per cm² of an area to be treated.

In some embodiments of any of the foregoing or following, said composition is exposed to actinic light, such as a LED light, having a wavelength between 400 nm and 700 nm, such as between 620 nm and 640 nm or between 425 nm and 450 nm. In certain such embodiments, the source of light has a minimal peak power density between 600-900 μW, such as about 719 μW or about 838 μW. In certain such embodiments, said composition is exposed for at least one treatment period of from about 1 minute to about 30 minutes per cm² of an area to be treated. In certain such embodiments, said composition is exposed to actinic light for at least one treatment period of from about 4 minutes to about 26 minutes, from about 8 minutes to about 24 minutes, from about 10 minutes to about 20 minutes, from about 10 minutes to about 18 minutes; or from about 1 minutes to about 5 minutes, from about 5 minutes to about 10 minutes, from about 10 minutes to about 15 minutes, from about 15 minutes to about 20 minutes, from about 20 minutes to about 25 minutes, or about 5 minutes, about 10 minutes, about 15 minutes, or about 20 minutes per cm² of an area to be treated.

In some embodiments of any of the foregoing or following, said composition is exposed to actinic light for at least one treatment period of from about 1 minute to about 9 minutes per cm² of an area to be treated. In certain such embodiments, said composition is exposed to actinic light for at least one treatment period of from about 2 minutes to about 8 minutes, from about 3 minutes to about 7 minutes, from about 4 minutes to about 6 minutes, or about 5 minutes per cm² of an area to be treated.

In some embodiments of any of the foregoing or following, said composition is exposed to actinic light for at least two treatment periods (e.g., two consecutive treatment periods). In certain such embodiments, said composition is exposed to actinic light for at least two treatment periods, each period followed by a resting interval.

In some embodiments of any of the foregoing or following, said composition is exposed to at least two treatment periods (e.g., two consecutive treatment periods) of actinic light wherein each treatment period is from about 1 minute to about 10 minutes per cm² of an area to be treated (e.g., from about 2 minutes to about 8 minutes, from about 3 minutes to about 7 minutes, from about 4 minutes to about 6 minutes, or about 5 minutes), wherein each treatment period is followed by a resting interval from about 1 minute to about 10 minutes (e.g., from about 1 minute to about 2 minutes, from about 1 minute to about 3 minutes, from about 2 minutes to about 8 minutes, from about 3 minutes to about 7 minutes, from about 4 minutes to about 6 minutes, from about 5 minutes to about 10 minutes, or about 5 minutes).

In some embodiments of any of the foregoing or following, said composition is exposed to at least two treatment periods (e.g., two consecutive treatment periods) of actinic light wherein each treatment period is from about 1 minute to about 5 minutes per cm² of an area to be treated, wherein each treatment period is followed by a resting interval from about 1 minute to about 10 minutes (e.g., from about 1 minute to about 2 minutes, from about 1 minute to about 3 minutes, from about 2 minutes to about 8 minutes, from about 3 minutes to about 7 minutes, from about 4 minutes to about 6 minutes, from about 5 minutes to about 10 minutes, or about 5 minutes).

In some embodiments of any of the foregoing or following, said composition is exposed to at least two treatment periods (e.g., two consecutive treatment periods) of actinic light wherein each treatment period is from about 1 minute to about 5 minutes per cm² of an area to be treated, wherein each treatment period is followed by a resting interval from about 5 minutes.

In some embodiments of any of the foregoing or following, said method further comprising:

a) topically applying the composition to the subject's area of pain;

b) exposing the subject's area of pain to actinic light for a period of from about 1 minute to about 10 minutes (e.g., from about 1 minute to 5 minutes or about 5 minutes);

c) removing the source of actinic light away from the subject's area of pain for a resting interval of from about 1 minute to about 10 minutes (e.g., from about 1 minute to 5 minutes or about 5 minutes);

d) exposing the subject's area of pain to actinic light for a second treatment period of from about 1 minute to about 10 minutes (e.g., from about 1 minute to 5 minutes or about 5 minutes); and wherein the first exposure to actinic light activates the composition.

In some embodiments of any of the foregoing or following, said method further comprising:

a) topically applying the composition to the subject's area of pain;

b) exposing the subject's area of pain to actinic light for a treatment period of from about 1 minute to about 10 minutes (e.g., from about 1 minute to 5 minutes or about 5 minutes);

c) removing the source of actinic light away from the subject's area of pain for a resting interval of from about 1 minute to about 5 minutes;

d) exposing the subject's area of pain to actinic light for a second treatment period of from about 1 minute to about 10 minutes (e.g., from about 1 minute to 5 minutes or about 5 minutes); and wherein the first exposure to actinic light activates the composition.

In some embodiments of any of the foregoing or following, the second exposure activates any residual composition.

In some embodiments of any of the foregoing or following, the method further comprises topically re-applying the composition before each treatment period, e.g., before the second treatment period.

In some embodiments of any of the foregoing or following, the source of actinic light is illuminating in continuous motion over an area to be treated.

In some embodiments of any of the foregoing or following, the source of actinic light is presented over an area to be treated. In some embodiments, said actinic light is visible light having a wavelength between about 400 nm and about 700 nm.

In some embodiments of any of the foregoing or following, the pain is acute or chronic pain (e.g., nociceptive pain or neuropathic pain).

In some embodiments of any of the foregoing or following, the pain is selected from the group consisting of widespread pain, localized pain, nociceptive pain, inflammatory pain, peripheral neuropathic pain, peripheral neurogenic pain, peripheral neuralgia, low back pain, postoperative pain, visceral pain, and pelvic pain; allodynia; anesthesia dolorosa; causalgia; dysesthesia; fibromyalgia; hyperalgesia; hyperesthesia; ischemic pain; sciatic pain; pain associated with cystitis including, but not limited to, interstitial cystitis; pain associated with multiple sclerosis; pain associated with arthritis; pain associated with osteoarthritis; pain associated with rheumatoid arthritis; pain associated with chronic wounds; pain associated with burns; and pain associated with cancer.

In some embodiments of any of the foregoing or following, the pain is associated with chronic wounds (e.g., venous leg ulcers or diabetic foot ulcers). In other embodiments, the pain is associated with acute wounds. In some embodiments, the pain is associated with burns. In other embodiments, the pain is associated with postoperative care. In some embodiments, the pain is associated with post-surgical wounds.

In some embodiments, the pain is reduced by at least 10%, at least 20%, at least 30%, at least 40%, at least 50%, at least 60%, at least 70%, at least 80%, at least 90%, or reduced fully. In some embodiments, the pain is reduced by at least one point, at least two points, at least three points, at least four points, at least five points, at least six points, at least seven points, at least eight points, or at least nine points on a standard pain score scale. As described herein, various methods of rating or scoring pain level is known in the art, including, but not limited to, a numerical analog score, a verbal assessment score, a verbal pain score, a visual analog score, etc. In certain embodiments, when using such a scoring scale, it is possible to measure a reduction in pain according to the scale (e.g., a reduction in 2 points if reduced from 10 to 8). In some embodiments, when using such a scoring scale, it is possible to measure a reduction in pain by percentage (e.g., as a percentage in reduction from a score of 10 to 8).

In some embodiments of any of the foregoing or following, the medical condition is associated with skin or soft tissues.

In some embodiments of any of the foregoing or following, the subject is a mammal, such as a human, an equine, a feline, or a canine.

In some aspects, the disclosure provides for use of a composition for the manufacture of a medicament for reducing pain that is associated with a medical condition in a subject, wherein said composition comprises at least one photoactivator and a pharmaceutically acceptable carrier; wherein the pain associated with the medical condition is treated non-systemically. In certain aspects, the composition further comprises at least one oxidant. In certain such aspects, the photoactivator is capable of activating the oxidant. In certain such aspects, the pain does not originate from a stimulus in an orofacial region. In certain such aspects, the reduction of pain is not dependent on the treatment of the underlying medical condition.

In some aspects, the disclosure provides for use of a composition for reducing pain that is associated with a medical condition in a subject, the composition comprising: at least one photoactivator and a pharmaceutically acceptable carrier, and wherein the pain associated with the medical condition is treated non-systemically. In certain such aspects, the composition further comprises at least one oxidant. In certain such aspects, the photoactivator is capable of activating the oxidant. In certain such aspects, the pain does not originate from a stimulus in an orofacial region. In certain such aspects, the reduction of pain is not dependent on the treatment of the underlying medical condition.

The compositions of the methods and uses of the present disclosure comprise a number of active principles selected from groups of possible components. These various active principles each have their mechanisms of action.

### Compositions

The present disclosure provides methods and uses comprising a composition, e.g., a composition for reducing or relieving pain. In one aspect, the composition of the present disclosure is a biophotonic composition. Compositions of this disclosure, in a broad sense, are activated by light (e.g., photons) of specific wavelength. These compositions contain at least one exogenous photoactivator or chromophore which is activated by light and accelerates the dispersion of light energy, which leads to light carrying on a therapeutic effect on its own, and/or to the photochemical activation of other agents contained in the composition. In some aspects, the disclosure provides a method of reducing pain that is associated with a medical condition in a subject, comprising: 1) topically applying a composition comprising at least one photoactivator, such as a photoactivator or chromophore; and exposing said composition to actinic light to cause activation of said composition, wherein the pain associated with the medical condition is treated non-systemically.

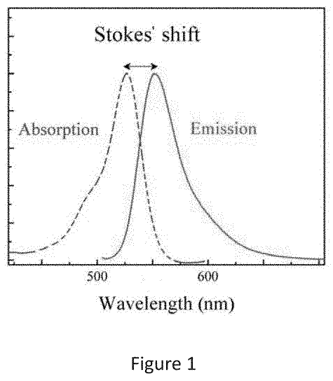

When a photoactivator or chromophore absorbs a photon of a certain wavelength, it becomes excited. This is an unstable condition and the molecule tries to return to the ground state, giving away the excess energy. For some photoactivators or chromophores, it is favorable to emit the excess energy as light when transforming back to the ground state. This process is called fluorescence. The peak wavelength of the emitted fluorescence is shifted towards longer wavelengths compared to the absorption wavelengths (‘Stokes’ shift’). The emitted fluorescent energy can then be transferred to the other components of the composition or to a treatment site on to which the composition is topically applied. Differing wavelengths of light may have different and complementary therapeutic effects on tissue. Stokes' shift is illustrated in FIG. 1.

In certain embodiments, the compositions of the present disclosure are substantially transparent/translucent and/or have high light transmittance in order to permit light dissipation into and through the composition. In this way, the area of tissue under the composition can be treated both with the fluorescent light emitted by the composition and the light irradiating the composition to activate it, which may benefit from the different therapeutic effects of light having different wavelengths.

The % transmittance of the composition can be measured in the range of wavelengths from 250 nm to 800 nm using, for example, a Perkin-Elmer Lambda 9500 series UV-visible spectrophotometer. Alternatively, a Synergy HT spectrophotometer (BioTek Instrument, Inc.) can be used in the range of wavelengths from 380 nm to 900 nm.

Transmittance is calculated according to the following equation:

$${A}_{λ} = {log}_{10} \frac{{I}_{0}}{I} = {log}_{10} \frac{1}{T} .$$

where A is absorbance, T is transmittance, I₀ is intensity of radiation before passing through material, and I is intensity of light passing through material.

The values can be normalized for thickness. As stated herein, % transmittance (translucency) is as measured for a 2 mm thick sample at a wavelength of 526 nm. It will be clear that other wavelengths can be used.

In certain embodiments of the disclosure, the compositions of the present disclosure are for topical uses. The composition can be in the form of a semi-solid or viscous liquid, such as a gel, or are gel-like, and which have a spreadable consistency at room temperature (e.g., about 20-25° C.) prior to illumination. In certain such embodiments wherein the composition has a spreadable consistency, the composition can be topically applied to a treatment site at a thickness of from about 0.5 mm to about 3 mm, from about 0.5 mm to about 2.5 mm, or from about 1 mm to about 2 mm. In some embodiments, the composition can be topically applied to a treatment site at a thickness of about 2 mm or about 1 mm. Spreadable compositions can conform to a topography of a treatment site. This can have advantages over a non-conforming material in that a better and/or more complete illumination of the treatment site can be achieved and the compositions are easy to apply and remove.

These compositions may be described based on the components making up the composition. Additionally or alternatively, the compositions of the present disclosure have functional and structural properties and these properties may also be used to define and describe the compositions. Individual components of the composition of the present disclosure are detailed as below.

### Oxidants

In certain embodiments, the compositions of the methods and uses of the present disclosure may comprise oxidants, such as, for example, peroxide compounds. Peroxide compounds are oxidants that contain the peroxy group (R—O—O—R), which is a chainlike structure containing two oxygen atoms, each of which is bonded to the other and a radical or some element. Suitable oxidants for preparation of the active medium include, but are not limited to:

Hydrogen peroxide (H₂O₂) is the starting material to prepare organic peroxides. H₂O₂ is a powerful oxidizing agent, and the unique property of hydrogen peroxide is that it breaks down into water and oxygen and does not form any persistent, toxic residual compound. Hydrogen peroxide for use in this composition can be used in a gel, for example with about 6% hydrogen peroxide by weight of the composition. A suitable range of concentration over which hydrogen peroxide can be used in the present composition is less than about 12% by weight of the compositions. In some embodiments, hydrogen peroxide is present in an amount of from about 0.1% to about 12%, from about 1% to about 12%, from about 3.5% to about 12%, from about 3.5% to about 6% or from about 0.1% to about 6% by weight of the total composition. In some embodiments, hydrogen peroxide is present in an amount from about 0.1% to about 12%, from about 0.1% to about 10%, from about 0.1% to about 8%, from about 0.1% to about 6% or from about 0.1% to about 4% by weight of the total composition.

Urea hydrogen peroxide (also known as urea peroxide, carbamide peroxide or percarbamide) is soluble in water and contains approximately 36% hydrogen peroxide. Carbamide peroxide for use in this composition can be used as a gel, for example with 16% carbamide peroxide that represents 5.6% hydrogen peroxide. A suitable range of concentration over which urea peroxide can be used in the present composition is less than 36% by weight of the total composition. In some embodiments, urea peroxide is present in an amount from about 0.3% to about 36%, or from about 3% to about 36%, or from about 10% to about 36%, or from about 3% to about 16% or from about 0.3% to about 16% by weight of the total composition. In some embodiments, urea peroxide is present in an amount of about 2% by weight of the total composition. In some embodiments, urea peroxide is present in an amount of about 3% by weight of the total composition. In some embodiments, urea peroxide is present in an amount of about 6% by weight of the total composition. In some embodiments, urea peroxide is present in an amount of about 8% by weight of the total composition. In some embodiments, urea peroxide is present in an amount of about 12% by weight of the total composition. Urea peroxide brakes down to urea and hydrogen peroxide in a slow-release fashion that can be accelerated with heat or photochemical reactions. The released urea (carbamide, (NH₂)CO₂), is highly soluble in water and is a powerful protein denaturant. It increases solubility of some proteins and enhances rehydration of the skin and/or mucosa.

Benzoyl peroxide consists of two benzoyl groups (benzoic acid with the H of the carboxylic acid removed) joined by a peroxide group. The released peroxide groups are effective as antibacterial agents. Benzoyl peroxide also promotes skin turnover and clearing of pores. Benzoyl peroxide breaks down to benzoic acid and oxygen upon contact with skin, neither of which is toxic. A suitable range of concentration over which benzoyl peroxide can be used in the present composition is less than about 10% by weight of the total composition. In some embodiments, benzoyl peroxide is present in an amount of from about 1% to about 10%, or from about 1% to about 8%, or from about 2.5% to about 5% by weight of the total composition.

Suitable oxidants may also include peroxy acids and alkali metal percarbonates, but the inclusion of any other forms of peroxides (e.g., organic or inorganic peroxides) should be avoided due to their increased toxicity and their unpredictable reaction with the photodynamic energy transfer.

### Photoactivators/Chromophores/Photoactivating Agents

The compositions, such as biophotonic topical compositions, of the methods and uses of the present disclosure comprise one or more photoactivators or chromophores, which can be considered exogenous, e.g., are not naturally present in skin or tissue. When a composition of the present disclosure is illuminated with light, the chromophore(s) are excited to a higher energy state. When the chromophore(s)' electrons return to a lower energy state, they emit photons with a lower energy level, thus causing the emission of light of a longer wavelength (Stokes' shift).

Suitable photoactivators or chromophores for the compositions of the disclosure can be fluorescent dyes (or stains), although other dye groups or dyes (biological and histological dyes, food colorings, carotenoids, naturally occurring fluorescent and other dyes) can also be used.

In some embodiments, the composition of the present disclosure comprises a chromophore which undergoes partial or complete photobleaching upon application of light. By photobleaching is meant a photochemical destruction of the chromophore which can generally be visualized as a loss of color.

In some embodiments, the chromophore absorbs at a wavelength in the range of the visible spectrum, such as at a wavelength of about 380-800 nm, about 380-700 nm, or about 380-600 nm. In some embodiments, the chromophore absorbs at a wavelength of about 200-800 nm, about 200-700 nm, about 200-600 nm or about 200-500 nm. In some embodiments, the chromophore absorbs at a wavelength of about 200-600 nm. In some embodiments, the chromophore absorbs light at a wavelength of about 200-300 nm, about 250-350 nm, about 300-400 nm, about 350-450 nm, about 400-500 nm, about 400-600 nm, about 450-650 nm, about 600-700 nm, about 650-750 nm or about 700-800 nm.

In some embodiments, the chromophore or combination of chromophores is present in an amount of about 0.001-40% by weight of the composition. In some embodiments, the chromophore or combination of chromophores is present in an amount of about 0.005-2%, about 0.01-1%, about 0.01-2%, about 0.05-1%, about 0.05-2%, about 0.1-1%, about 0.1-2%, about 1-5%, about 2.5-7.5%, about 5-10%, about 7.5-12.5%, about 10-15%, about 12.5-17.5%, about 15-20%, about 17.5-22.5%, about 20-25%, about 22.5-27.5%, about 25-30%, about 27.5-32.5%, about 30-35%, about 32.5-37.5%, or about 35-40% by weight of the composition. In some embodiments, the chromophore or combination of chromophores is present in an amount of at least about 0.2% by weight of the composition.

In some embodiments, the chromophore or combination of chromophores is present in an amount of 0.001-40% by weight of the composition. In some embodiments, the chromophore or combination of chromophores is present in an amount of 0.005-2%, 0.01-1%, 0.01-2%, 0.05-1%, 0.05-2%, 0.1-1%, 0.1-2%, 1-5%, 2.5-7.5%, 5-10%, 7.5-12.5%, 10-15%, 12.5-17.5%, 15-20%, 17.5-22.5%, 20-25%, 22.5-27.5%, 25-30%, 27.5-32.5%, 30-35%, 32.5-37.5%, or 35-40% by weight of the composition. In some embodiments, the chromophore or combination of chromophores is present in an amount of at least 0.2% by weight of the composition.

It will be appreciated to those skilled in the art that optical properties of a particular chromophore may vary depending on the chromophore's surrounding medium. Therefore, as used herein, a particular chromophore's absorption and/or emission wavelength (or spectrum) corresponds to the wavelengths (or spectra) measured in a composition of the present disclosure.

The compositions disclosed herein may include at least one additional chromophore.

Combining chromophores may increase photo-absorption by the combined dye molecules and enhance absorption and photo-biomodulation selectivity. This creates multiple possibilities of generating new photosensitive, and/or selective chromophores mixtures.

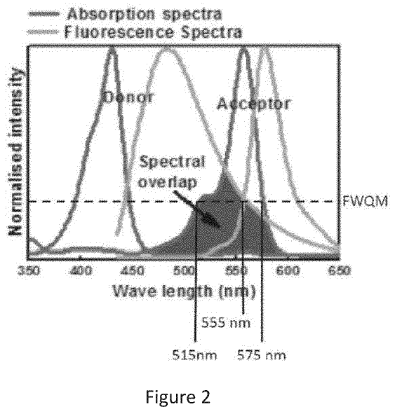

When such multi-chromophore compositions are illuminated with light, energy transfer can occur between the chromophores. This process, known as resonance energy transfer, is a photophysical process through which an excited ‘donor’ chromophore (also referred to herein as first chromophore) transfers its excitation energy to an ‘acceptor’ chromophore (also referred to herein as second chromophore). The efficiency and directedness of resonance energy transfer depends on the spectral features of donor and acceptor chromophores. In particular, the flow of energy between chromophores is dependent on a spectral overlap reflecting the relative positioning and shapes of the absorption and emission spectra. For energy transfer to occur the emission spectrum of the donor chromophore overlap with the absorption spectrum of the acceptor chromophore (FIG. 2).

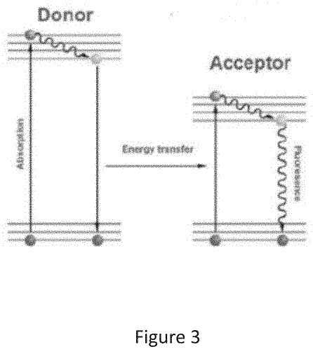

Energy transfer manifests itself through decrease or quenching of the donor emission and a reduction of excited state lifetime accompanied also by an increase in acceptor emission intensity. FIG. 3 is a Jablonski diagram that illustrates the coupled transitions involved between a donor emission and acceptor absorbance.

To enhance the energy transfer efficiency, the donor chromophore should have good abilities to absorb photons and emit photons. Furthermore, it is thought that the more overlap there is between the donor chromophore's emission spectra and the acceptor chromophore's absorption spectra, the better a donor chromophore can transfer energy to the acceptor chromophore.

In some embodiments, the biophotonic topical composition of the present disclosure further comprises an acceptor, or a second, chromophore. In some embodiments, the donor, or first, chromophore has an emission spectrum that overlaps at least about 80%, about 70%, about 60%, about 50%, about 40%, about 30%, about 20%, or about 10% with an absorption spectrum of the second chromophore. In some embodiments, the first chromophore has an emission spectrum that overlaps at least about 20% with an absorption spectrum of the second chromophore. In some embodiments, the first chromophore has an emission spectrum that overlaps at least 1-10%, 5-15%, 10-20%, 15-25%, 20-30%, 25-35%, 30-40%, 35-45%, 50-60%, 55-65% or 60-70% with an absorption spectrum of the second chromophore.

% spectral overlap, as used herein, refers to the % overlap of a donor chromophore's emission wavelength range with an acceptor chromophore's absorption wavelength range, measured at spectral full width quarter maximum (FWQM). For example, FIG. 2 shows the normalized absorption and emission spectra of donor and acceptor chromophores. The spectral FWQM of the acceptor chromophore's absorption spectrum is from about 60 nm (about 515 nm to about 575 nm). The overlap of the donor chromophore's spectrum with the absorption spectrum of the acceptor chromophore is about 40 nm (from 515 nm to about 555 nm). Thus, the % overlap can be calculated as 40 nm/60 nm×100=66.6%.

In some embodiments, the second chromophore absorbs at a wavelength in the range of the visible spectrum. In some embodiments, the second chromophore has an absorption wavelength that is relatively longer than that of the first chromophore within the range of about 50-250 nm, about 25-150 nm or about 10-100 nm.

As discussed above, the application of light to the compositions of the present disclosure can result in a cascade of energy transfer between the chromophores. In some embodiments, such a cascade of energy transfer provides photons that penetrate the epidermis, dermis and/or mucosa at the target tissue, including, such as, a site of wound.

In some embodiments, the chromophore or chromophores are selected such that their emitted fluorescent light, on photoactivation, is within one or more of the green, yellow, orange, red and infrared portions of the electromagnetic spectrum, for example having a peak wavelength within the range of about 490 nm to about 800 nm. In some embodiments, the emitted fluorescent light has a power density of between 0.005 to about 10 mW/cm², about 0.5 to about 5 mW/cm².

Suitable chromophores useful in the compositions (such as the biophotonic compositions), methods, and uses of the present disclosure include, but are not limited to the following:

### Xanthene Derivatives

The xanthene derivative dyes have been used and tested for a long time worldwide. They display low toxicity and increased fluorescence. The xanthene group consists of three sub-groups: a) the fluorenes; b) fluorones; and c) the rhodoles, any of which may be suitable for the compositions, methods, and uses of the present disclosure.

The fluorenes group comprises the pyronines (e.g., pyronine Y and B) and the rhodamines (e.g., rhodamine B, G and WT). Depending on the concentration used, both pyronines and rhodamines may be toxic and their interaction with light may lead to increased toxicity. Similar effects are known to occur for the rhodole dye group.

The fluorone group comprises the fluorescein dye and the fluorescein derivatives.

Fluorescein is a fluorophore commonly used in microscopy with an absorption maximum of 494 nm and an emission maximum of 521 nm. The disodium salt of fluorescein is known as D&C Yellow 8. It has very high fluorescence but photodegrades quickly. In the present composition, mixtures of fluorescein with other photoactivators such as indocyanin green and/or saffron red powder will confer increased photoabsorption to these other compounds.

The eosins group comprises Eosin Y (tetrabromofluorescein, acid red 87, D&C Red 22), a chromophore with an absorption maximum of 514-518 nm that stains the cytoplasm of cells, collagen, muscle fibers and red blood cells intensely red; and Eosin B (acid red 91, eosin scarlet, dibromo-dinitrofluorescein), with the same staining characteristics as Eosin Y. Eosin Y and Eosin B are collectively referred to as “Eosin”, and use of the term “Eosin” refers to either Eosin Y, Eosin B or a mixture of both. Eosin Y, Eosin B, or a mixture of both can be used because of their sensitivity to the light spectra used: broad spectrum blue light, blue to green light and green light. In some embodiments, the composition includes in the range of less than about 12% by weight of the total composition of at least one of Eosin B or Eosin Y or a combination thereof. In some embodiments, at least one of Eosin B or Eosin Y or a combination thereof is present from about 0.001% to about 12%, or between about 0.01% and about 1.2%, or from about 0.01% to about 0.5%, or from about 0.01% to about 0.05%, or from about 0.1% to about 0.5%, or from about 0.5% to about 0.8% by weight of the total composition. In some embodiments, at least one of Eosin B or Eosin Y or a combination thereof is present is an amount of at about 0.005% by weight of the total composition. In some embodiments, at least one of Eosin B or Eosin Y or a combination thereof is present is an amount of at about 0.01% by weight of the total composition. In some embodiments, at least one of Eosin B or Eosin Y or a combination thereof is present is an amount of at about 0.02% by weight of the total composition. In some embodiments, at least one of Eosin B or Eosin Y or a combination thereof is present is an amount of at about 0.05% by weight of the total composition. In some embodiments, at least one of Eosin B or Eosin Y or a combination thereof is present is an amount of at about 0.1% by weight of the total composition. In some embodiments, at least one of Eosin B or Eosin Y or a combination thereof is present is an amount of at about 0.2% by weight of the total composition. In some embodiments, at least one of Eosin B or Eosin Y or a combination thereof is present is an amount of at least about 0.2% by weight of the total composition but less than about 1.2% by weight of the total composition. In some embodiments, at least one of Eosin B or Eosin Y or a combination thereof is present is an amount of at least about 0.01% by weight of the total composition but less than about 12% by weight of the total composition.

In some embodiments, the composition includes in the range of less than 12% by weight of the total composition of at least one of Eosin B or Eosin Y or a combination thereof. In some embodiments, at least one of Eosin B or Eosin Y or a combination thereof is present from 0.001% to 12%, or between 0.01% and 1.2%, or from 0.01% to 0.5%, or from 0.1% to 0.5%, or from 0.5% to 0.8%, or from 0.01% to 0.05%, by weight of the total composition. In some embodiments, at least one of Eosin B or Eosin Y or a combination thereof is present is an amount of at 0.005% by weight of the total composition. In some embodiments, at least one of Eosin B or Eosin Y or a combination thereof is present is an amount of at 0.01% by weight of the total composition. In some embodiments, at least one of Eosin B or Eosin Y or a combination thereof is present is an amount of at 0.02% by weight of the total composition. In some embodiments, at least one of Eosin B or Eosin Y or a combination thereof is present is an amount of at 0.05% by weight of the total composition. In some embodiments, at least one of Eosin B or Eosin Y or a combination thereof is present is an amount of at 0.1% by weight of the total composition. In some embodiments, at least one of Eosin B or Eosin Y or a combination thereof is present is an amount of at 0.2% by weight of the total composition. In some embodiments, at least one of Eosin B or Eosin Y or a combination thereof is present is an amount of at least 0.2% by weight of the total composition but less than 1.2% by weight of the total composition. In some embodiments, at least one of Eosin B or Eosin Y or a combination thereof is present is an amount of at least 0.01% by weight of the total composition but less than 12% by weight of the total composition.

Phloxine B (2,4,5,7 tetrabromo 4,5,6,7,tetrachlorofluorescein, D&C Red 28, acid red 92) is a red dye derivative of fluorescein which is used for disinfection and detoxification of waste water through photooxidation. It has an absorption maximum of 535-548 nm. It is also used as an intermediate for making photosensitive dyes and drugs.

Erythrosine B, or simply Erythrosine or Erythrosin (acid red 51, tetraiodofluorescein) is a cherry-pink, coal-based fluorine food dye used as a biological stain, and a biofilm and dental plaque disclosing agent, with a maximum absorbance of 524-530 nm in aqueous solution. It is subject to photodegradation. Erythrosine is also used in some embodiments due to its photosensitivity to the light spectra used and its ability to stain biofilms. In embodiments, the composition includes in the range of less than about 2% by weight Erythrosine B. In some embodiments, Erythrosine B is present in an amount from about 0.005 to about 2%, or from about 0.005% to about 1%, or about 0.01% to about 1% by weight of the total composition. In some embodiments, Erythrosine B is present in an amount of about 0.005% and about 0.15% by weight of the total composition.

Rose Bengal (4,5,6,7 tetrachloro 2,4,5,7 tetraiodofluorescein, acid red 94) is a bright bluish-pink fluorescein derivative with an absorption maximum of 544-549 nm, that has been used as a dye, biological stain and diagnostic aid.

Merbromine (mercurochrome) is an organo-mercuric disodium salt of fluorescein with an absorption maximum of 508 nm. It is used as an antiseptic.

### Azo Dyes

The azo (or diazo-) dyes share the N—N group, called azo the group. They are used mainly in analytical chemistry or as food colorings and are not fluorescent. Suitable azo dyes for the compositions, methods, and uses of the disclosure include: Methyl violet, neutral red, para red (pigment red 1), amaranth (Azorubine S), Carmoisine (azorubine, food red 3, acid red 14), allura red AC (FD&C 40), tartrazine (FD&C Yellow 5), orange G (acid orange 10), Ponceau 4R (food red 7), methyl red (acid red 2), and murexide-ammonium purpurate.

### Biological Stains

Dye molecules commonly used in staining protocols for biological materials can also be used as photoactivators for the compositions, methods, and uses of the disclosure. Suitable biological stains include:

Safranin (Safranin 0, basic red 2) is an azo-dye and is used in histology and cytology. It is a classic counter stain in a Gram stain protocol.

Fuchsin (basic or acid) (rosaniline hydrochloride) is a magenta biological dye that can stain bacteria and has been used as an antiseptic. It has an absorption maximum of 540-555 nm.

3,3′ dihexylocarbocyanine iodide (DiOC6) is a fluorescent dye used for staining the endoplasmic reticulum, vesicle membranes and mitochondria of cells. It shows photodynamic toxicity; when exposed to blue light, has a green fluorescence.

Carminic acid (acid red 4, natural red 4) is a red glucosidal hydroxyanthrapurin naturally obtained from cochineal insects.

Indocyanin green (ICG) is used as a diagnostic aid for blood volume determination, cardiac output, or hepatic function. ICG binds strongly to red blood cells and when used in mixture with fluorescein, it increases the absorption of blue to green light.

### Carotenoids

Carotenoid dyes are also photoactivators that are useful in the compositions, methods, and uses of the disclosure.

Saffron red powder is a natural carotenoid-containing compound. Saffron is a spice derived from crocus sativus. It is characterized by a bitter taste and iodoform or hay-like fragrance; these are caused by the compounds picrocrocin and saffranal. It also contains the carotenoid dye crocin that gives its characteristic yellow-red color.

Saffron contains more than 150 different compounds, many of which are carotenoids: mangicrocin, reaxanthine, lycopene, and various α and β-carotenes, which show good absorption of light and beneficial biological activity. Also saffron can act as both a photon-transfer agent and a healing factor. Saffron color is primarily the result of a-crocin (8,8 diapo-8,8-carotenoid acid). Dry saffron red powder is highly sensitive to fluctuating pH levels and rapidly breaks down chemically in the presence of light and oxidizing agents. It is more resistant to heat. Data show that saffron has anticarcinogenic, immunomodulating and antioxidant properties. For absorbance, the crocin specific photon wavelength is 440 nm (blue light). It has a deep red colour and forms crystals with a melting point of 186° C. When dissolved in water, it forms an orange solution.

Crocetin, another compound of saffron, was found to express an antilipidemic action and promote oxygen penetration in different tissues. More specifically, an increased oxygenation of the endothelial cells of the capillaries was observed. Additionally, an increase of the oxygenation of muscles and cerebral cortex was observed and led to an improved survival rate in laboratory animals with induced hemorrhagic shock or emphysema.

Anatto, a spice, contains as main constituent (70-80%) the carotenoid bixin which displays relevant antioxidative properties. β-carotene, also displays suitable characteristics.

Fucoxanthine is a constituent of brown algae with a pronounced ability for photosensitization of redox reactions.

### Chlorophyll Dyes

Exemplary chlorophyll dyes that are useful in the compositions, methods, and uses of the disclosure, include but are not limited to chlorophyll a, chlorophyll b, oil soluble chlorophyll, bacteriochlorophyll a, bacteriochlorophyll b, bacteriochlorophyll c, bacteriochlorophyll d, protochlorophyll, protochlorophyll a, amphiphilic chlorophyll derivative 1, and amphiphilic chlorophyll derivative 2.

In some aspects of the disclosure, the one or more chromophores of the composition disclosed herein can be independently selected from any of Acid black 1, Acid blue 22, Acid blue 93, Acid fuchsin, Acid green, Acid green 1, Acid green 5, Acid magenta, Acid orange 10, Acid red 26, Acid red 29, Acid red 44, Acid red 51, Acid red 66, Acid red 87, Acid red 91, Acid red 92, Acid red 94, Acid red 101, Acid red 103, Acid roseine, Acid rubin, Acid violet 19, Acid yellow 1, Acid yellow 9, Acid yellow 23, Acid yellow 24, Acid yellow 36, Acid yellow 73, Acid yellow S, Acridine orange, Acriflavine, Alcian blue, Alcian yellow, Alcohol soluble eosin, Alizarin, Alizarin blue 2RC, Alizarin carmine, Alizarin cyanin BBS, Alizarol cyanin R, Alizarin red S, Alizarin purpurin, Aluminon, Amido black 10B, Amidoschwarz, Aniline blue WS, Anthracene blue SWR, Auramine O, Azocannine B, Azocarmine G, Azoic diazo 5, Azoic diazo 48, Azure A, Azure B, Azure C, Basic blue 8, Basic blue 9, Basic blue 12, Basic blue 15, Basic blue 17, Basic blue 20, Basic blue 26, Basic brown 1, Basic fuchsin, Basic green 4, Basic orange 14, Basic red 2 (Safranin O), Basic red 5, Basic red 9, Basic violet 2, Basic violet 3, Basic violet 4, Basic violet 10, Basic violet 14, Basic yellow 1, Basic yellow 2, Biebrich scarlet, Bismarck brown Y, Brilliant crystal scarlet 6R, Calcium red, Carmine, Carminic acid (acid red 4), Celestine blue B, China blue, Cochineal, Celestine blue, Chrome violet CG, Chromotrope 2R, Chromoxane cyanin R, Congo corinth, Congo red, Cotton blue, Cotton red, Croceine scarlet, Crocin, Crystal ponceau 6R, Crystal violet, Dahlia, Diamond green B, DiOC6, Direct blue 14, Direct blue 58, Direct red, Direct red 10, Direct red 28, Direct red 80, Direct yellow 7, Eosin B, Eosin Bluish, Eosin, Eosin Y, Eosin yellowish, Eosinol, Erie garnet B, Eriochrome cyanin R, Erythrosin B, Ethyl eosin, Ethyl green, Ethyl violet, Evans blue, Fast blue B, Fast green FCF, Fast red B, Fast yellow, Fluorescein, Food green 3, Gallein, Gallamine blue, Gallocyanin, Gentian violet, Haematein, Haematine, Haematoxylin, Helio fast rubin BBL, Helvetia blue, Hematein, Hematine, Hematoxylin, Hoffman's violet, Imperial red, Indocyanin green, Ingrain blue, Ingrain blue 1, Ingrain yellow 1, INT, Kermes, Kermesic acid, Kernechtrot, Lac, Laccaic acid, Lauth's violet, Light green, Lissamine green SF, Luxol fast blue, Magenta 0, Magenta I, Magenta II, Magenta III, Malachite green, Manchester brown, Martius yellow, Merbromin, Mercurochrome, Metanil yellow, Methylene azure A, Methylene azure B, Methylene azure C, Methylene blue, Methyl blue, Methyl green, Methyl violet, Methyl violet 2B, Methyl violet 10B, Mordant blue 3, Mordant blue 10, Mordant blue 14, Mordant blue 23, Mordant blue 32, Mordant blue 45, Mordant red 3, Mordant red 11, Mordant violet 25, Mordant violet 39 Naphthol blue black, Naphthol green B, Naphthol yellow S, Natural black 1, Natural red, Natural red 3, Natural red 4, Natural red 8, Natural red 16, Natural red 25, Natural red 28, Natural yellow 6, NBT, Neutral red, New fuchsin, Niagara blue 3B, Night blue, Nile blue, Nile blue A, Nile blue oxazone, Nile blue sulphate, Nile red, Nitro BT, Nitro blue tetrazolium, Nuclear fast red, Oil red O, Orange G, Orcein, Pararosanilin, Phloxine B, phycobilins, Phycocyanins, Phycoerythrins. Phycoerythrincyanin (PEC), Phthalocyanines, Picric acid, Ponceau 2R, Ponceau 6R, Ponceau B, Ponceau de Xylidine, Ponceau S, Primula, Purpurin, Pyronin B, Pyronin G, Pyronin Y, Rhodamine B, Rosanilin, Rose bengal, Saffron, Safranin O, Scarlet R, Scarlet red, Scharlach R, Shellac, Sirius red F3B, Solochrome cyanin R, Soluble blue, Solvent black 3, Solvent blue 38, Solvent red 23, Solvent red 24, Solvent red 27, Solvent red 45, Solvent yellow 94, Spirit soluble eosin, Sudan III, Sudan IV, Sudan black B, Sulfur yellow S, Swiss blue, Tartrazine, Thioflavine S, Thioflavine T, Thionin, Toluidine blue, Toluyline red, Tropaeolin G, Trypaflavine, Trypan blue, Uranin, Victoria blue 4R, Victoria blue B, Victoria green B, Water blue I, Water soluble eosin, Xylidine ponceau, or Yellowish eosin.

In some embodiments, the composition includes Eosin Y as a first chromophore. In some embodiments, the composition includes Eosin Y as a first chromophore and any one or more of Rose Bengal, Erythrosin, Phloxine B as a second chromophore.

In some embodiments, the composition includes the following synergistic combinations: Eosin Y and Fluorescein; Fluorescein and Rose Bengal; Erythrosine in combination with one or more of Eosin Y, Rose Bengal or Fluorescein; or Phloxine B in combination with one or more of Eosin Y, Rose Bengal, Fluorescein and Erythrosine. Other synergistic chromophore combinations are also possible.

By means of synergistic effects of the chromophore combinations in the composition, chromophores which cannot normally be activated by an activating light (such as a blue light from an LED) can be activated through energy transfer from chromophores which are activated by the activating light. In this way, the different properties of photoactivated chromophores can be harnessed and tailored according to the cosmetic or the medical therapy required.

Chromophore combinations can also have a synergistic effect in terms of their photoactivated state. For example, two chromophores may be used, one of which emits fluorescent light when activated in the blue and green range, and the other which emits fluorescent light in the red, orange and yellow range, thereby complementing each other and irradiating the target tissue with a broad wavelength of light having different depths of penetration into target tissue and different therapeutic effects.

### Healing Factors

Healing factors comprise compounds that promote or enhance the healing or regenerative process of the tissues on the application site of the composition. During the photoactivation of the composition, there is an increase of the absorption of molecules at the treatment site. An augmentation in the blood flow at the site of treatment is observed for an extent period of time. An increase in the lymphatic drainage and a possible change in the osmotic equilibrium due to the dynamic interaction of the free radical cascades can be enhanced or even fortified with the inclusion of healing factors. Suitable healing factors for the compositions, methods and uses of the present disclosure include, but are not limited to:

### Hyaluronic Acid (Hyaluronan or Hyaluronate)

Hyaluronic acid (hyaluronan or hyaluronate) is a non-sulfated glycosaminoglycan, distributed widely throughout connective, epithelial and neural tissues. It is one of the primary components of the extracellular matrix, and contributes significantly to cell proliferation and migration. Hyaluronan is a major component of the skin, where it is involved in tissue repair. While it is abundant in extracellular matrices, it contributes to tissue hydrodynamics, movement and proliferation of cells and participates in a wide number of cell surface receptor interactions, notably those including primary receptor CD44. The hyaluronidase enzymes degrade hyaluronan, and there are at least seven types of hyaluronidase-like enzymes in humans, several of which are tumor suppressors. The degradation products of hyaluronic acid, the oligosaccharides and the very-low molecular weight hyaluronic acid, exhibit pro-angiogenic properties. In addition, recent studies show that hyaluronan fragments, but not the native high molecular mass of hyaluronan, can induce inflammatory responses in macrophages and dendritic cells in tissue injury. Hyaluronic acid is well suited to biological applications targeting the skin. Due to its high biocompatibility, it is used to stimulate tissue regeneration. Current studies evidenced hyaluronic acid appearing in the early stages of healing to physically create room for white blood cells that mediate the immune response. It is used in the synthesis of biological scaffolds for wound healing applications and in wrinkle treatment. In certain embodiments, the composition includes hyaluronic acid in the range of less than about 2% by weight of the total composition hyaluronic acid. In some embodiments, hyaluronic acid is present in an amount from about 0.001% to about 2%, or from about 0.002% to about 2%, or from about 0.002% to about 1% by weight of the total composition.

### Glucosamine

Glucosamine is one of the most abundant monosaccharides in human tissues and a precursor in the biological synthesis of glycosylated proteins and lipids. It is commonly used in the treatment of osteoarthritis. The common form of glucosamine used is its sulfate salt. Glucosamine shows a number of effects, including anti-inflammatory activity, stimulation of the synthesis of proteoglycans and the synthesis of proteolytic enzymes. A suitable range of concentration over which glucosamine can be used in the present composition is from less than about 5% by weight of the total composition. In some embodiments, glucosamine is present in an amount from about 0.0001% to about 5%, or from about 0.0001% to about 3%, or from about 0.001% to about 3%, or from about 0.001% to about 1%, or from about 0.01% to about 1%, or from about 1% to about 3% by weight of the total composition.

### Allantoin

Allantoin is a diureide of glyosilic acid. It has keratolytic effect, increases the water content of the extracellular matrix, enhances the desquamation of the upper layers of dead (apoptotic) skin cells, and promotes skin proliferation and wound healing. In certain embodiments, the composition includes in the range of less than about 1% by weight of the total composition allantoin. In some embodiments, allantoin is present in an amount from about 0.001% to about 1%, or from about 0.002% to about 1%, or from about 0.02% to about 1%, or from about 0.02% to about 0.5% by weight of the total composition.

Also, saffron can act as both a photon-transfer agent and a healing factor.

### Chelating Agents

Chelating agents can be included to promote smear layer removal in closed pockets and difficult to reach lesions. Suitable chelating agents for the compositions, methods and uses of the disclosure include, but are not limited to:

### Ethylenediaminotetraacetic Acid (EDTA)

Ethylenediaminotetraacetic acid (EDTA) is an amino acid and is used to sequester di- and trivalent metal ions. EDTA binds to metals via four carboxylate and two amine groups. EDTA forms especially strong complexes with Mn(III), Fe(III), Cu(III), Co(III). It is used to buffer solutions.

### Ethylene Glycol Tetraacetic Acid (EGTA)

Ethylene glycol tetraacetic acid (EGTA) is related to EDTA, but with a much higher affinity for calcium than magnesium ions. It is useful for making buffer solutions that resemble the environment inside living cells.

### Carbonate and Bicarbonate Salts

According to some embodiments, the compositions of the present disclosure may optionally further comprise one or more carbonate or bicarbonate salts.

Suitable carbonate or bicarbonate salts that may be present in the composition include, but are not limited to: ammonium bicarbonate, caesium bicarbonate, potassium bicarbonate, sodium bicarbonate, choline bicarbonate, aminoguanidine bicarbonate, tetraethylammonium bicarbonate, barium carbonate, beryllium carbonate, caesium carbonate, calcium carbonate, cobalt (II) carbonate, copper (II) carbonate, lithium carbonate, magnesium carbonate, nickel (II) carbonate, potassium carbonate, sodium carbonate, or zinc carbonate.

In some embodiments, the composition of the disclosure comprises one or more salts selected from bicarbonate salts, carbonate salts or a combination of the foregoing salts. In some embodiments, the composition of the disclosure comprises one or more bicarbonate salts. In some embodiments when the composition comprises one or more bicarbonate salts, the bicarbonate salt is sodium bicarbonate. In some embodiments when the composition comprises one or more bicarbonate salts, the bicarbonate salt is potassium bicarbonate. In some embodiments, the composition of the disclosure comprises one or more carbonate salts. In some embodiments when the composition comprises one or more carbonate salts, the carbonate salt is sodium carbonate. In some embodiments when the composition comprises one or more carbonate salts, the carbonate salt is potassium carbonate. In some embodiments when the composition comprises one or more carbonate salts, the carbonate salt is calcium carbonate.

### Gelling Agents

Gelling agents for the compositions, uses or methods according to the present disclosure may comprise any ingredient suitable for use in composition as described herein. The gelling agent may be an agent capable of forming a cross-linked matrix, including physical and/or chemical cross-links. The gelling agent can be biocompatible, and may be biodegradable. In some embodiments, the gelling agent is able to form a hydrogel or a hydrocolloid. An appropriate gelling agent is one that can form a viscous liquid or a semisolid. In some embodiments, the gelling agent and/or the composition has appropriate light transmission properties. It is also important to select a gelling agent which will allow biophotonic activity of the chromophore(s). For example, some chromophores require a hydrated environment in order to fluoresce. The gelling agent may be able to form a gel by itself or in combination with other ingredients such as water or another gelling agent, or when applied to a treatment site, or when illuminated with light.

The gelling agent according to various embodiments of the present disclosure may include, but not be limited to, polyalkylene oxides, particularly polyethylene glycol and poly(ethylene oxide)-poly(propylene oxide) copolymers, including block and random copolymers; polyols such as glycerol, polyglycerol (particularly highly branched polyglycerol), propylene glycol and trimethylene glycol substituted with one or more polyalkylene oxides, e.g., mono-, di- and tri-polyoxyethylated glycerol, mono- and di-polyoxy-ethylated propylene glycol, and mono- and di-polyoxyethylated trimethylene glycol; polyoxyethylated sorbitol, polyoxyethylated glucose; acrylic acid polymers and analogs and copolymers thereof, such as polyacrylic acid per se, polymethacrylic acid, poly(hydroxyethylmethacrylate), poly(hydroxyethylacrylate), poly(methylalkylsulfoxide methacrylate), poly(methylalkylsulfoxide acrylate) and copolymers of any of the foregoing, and/or with additional acrylate species such as aminoethyl acrylate and mono-2-(acryloxy)-ethyl succinate; polymaleic acid; poly(acrylamides) such as polyacrylamide per se, poly(methacrylamide), poly(dimethylacrylamide), and poly(N-isopropyl-acrylamide); poly(olefinic alcohol)s such as poly(vinyl alcohol); poly(N-vinyl lactams) such as poly(vinyl pyrrolidone), poly(N-vinyl caprolactam), and copolymers thereof, polyoxazolines, including poly(methyloxazoline) and poly(ethyloxazoline); silicones, polyvinyl silicates, tetramethoxyorthosilicates, methyltrimethoxyorthosilicates, tetraalkoxyorthosilicates, trialkoxyorthosilicates, pressure sensitive silicone adhesives (such as BioPSA from Dow-Corning), and polyvinylamines.

The gelling agent according to some embodiments of the present disclosure may include a polymer selected from any of synthetic or semi-synthetic polymeric materials, polyacrylate copolymers, cellulose derivatives and polymethyl vinyl ether/maleic anhydride copolymers. In some embodiments, the hydrophilic polymer comprises a polymer that is a high molecular weight (i.e., molar masses of more than about 5,000, and in some instances, more than about 10,000, or about 100,000, or about 1,000,000) and/or cross-linked polyacrylic acid polymer.

In some embodiments, the gelling agent comprises a carbomer. Carbomers are synthetic high molecular weight polymer of acrylic acid that are cross-linked with either allylsucrose or allylethers of pentaerythritol having a molecular weight of about 3×10⁶. The gelation mechanism depends on neutralization of the carboxylic acid moiety to form a soluble salt. The polymer is hydrophilic and produces sparkling clear gels when neutralized. Carbomer gels possess good thermal stability in that gel viscosity and yield value are essentially unaffected by temperature. As a topical product, carbomer gels possess optimum rheological properties. The inherent pseudoplastic flow permits immediate recovery of viscosity when shear is terminated and the high yield value and quick break make it ideal for dispensing. Aqueous solution of Carbopol® is acidic in nature due to the presence of free carboxylic acid residues. Neutralization of this solution cross-links and gelatinizes the polymer to form a viscous integral structure of desired viscosity.

Carbomers are available as fine white powders which disperse in water to form acidic colloidal suspensions (a 1% dispersion has a pH of approximately 3) of low viscosity. Neutralization of these suspensions using a base, for example sodium, potassium or ammonium hydroxides, low molecular weight amines and alkanolamines, results in the formation of translucent gels. Nicotine salts such as nicotine chloride form stable water-soluble complexes with carbomers at about pH 3.5 and are stabilized at an optimal pH of about 5.6.

In some embodiments of the disclosure, the carbomer is Carbopol®. Such polymers are commercially available from B.F. Goodrich or Lubrizol under the designation Carbopol® 71G NF, 420, 430, 475, 488, 493, 910, 934, 934P, 940, 971PNF, 974P NF, 980 NF, 981 NF and the like. Carbopols are versatile controlled-release polymers, as described by Brock (Pharmacotherapy, 14:430-7 (1994), incorporated herein by reference) and Durrani (Pharmaceutical Res. (Supp.) 8:S-135 (1991), incorporated herein by reference), and belong to a family of carbomers which are synthetic, high molecular weight, non-linear polymers of acrylic acid, crosslinked with polyalkenyl polyether. In some embodiments, the carbomer is Carbopol® 974P NF, 980 NF, 5984 EP, ETD 2020NF, Ultrez 10 NF, 934 NF, 934P NF or 940 NF. In some embodiments, the carbomer is Carbopol® 980 NF, ETD 2020 NF, Ultrez 10 NF, Ultrez 21 or 1382 Polymer, 1342 NF, 940 NF. In some embodiments, about 0.05% to about 10%, about 0.5% to about 5%, or about 1% to about 3% by weight of the final composition of a high molecular weight carbopol can be present as the gelling agent. In some embodiments, the composition of the disclosure comprises about 0.05% to about 10%, about 0.5% to about 5%, or about 1% to about 3% by weight of the final composition of a high molecular weight carbopol.

In some embodiments, the gelling agent comprises a hygroscopic and/or a hydrophilic material useful for their water attracting properties. The hygroscopic or hydrophilic material may include, but is not limited to, glucosamine, glucosamine sulfate, polysaccharides, cellulose derivatives (hydroxypropyl methylcellulose, hydroxyethyl cellulose, hydroxypropyl cellulose, methylcellulose and the like), noncellulose polysaccharides (galactomannans, guar gum, carob gum, gum arabic, sterculia gum, agar, alginates and the like), glycosaminoglycan, poly(vinyl alcohol), poly(2-hydroxyethylmethylacrylate), polyethylene oxide, collagen, chitosan, alginate, a poly(acrylonitrile)-based hydrogel, poly(ethylene glycol)/poly(acrylic acid) interpenetrating polymer network hydrogel, polyethylene oxide-polybutylene terephthalate, hyaluronic acid, high-molecular-weight polyacrylic acid, poly(hydroxy ethylmethacrylate), poly(ethylene glycol), tetraethylene glycol diacrylate, polyethylene glycol methacrylate, and poly(methyl acrylate-co-hydroxyethyl acrylate). In some embodiments, the hydrophilic gelling agent is selected from glucose, modified starch, methyl cellulose, carboxymethyl cellulose, propyl cellulose, hydroxypropyl cellulose, carbomers, alginic acid, sodium alginate, potassium alginate, ammonium alginate, calcium alginate, agar, carrageenan, locust bean gum, pectin, and gelatin.

The gelling agent may be protein-based/naturally derived material such as sodium hyaluronate, gelatin or collagen, lipids, or the like. The gelling agent may be a polysaccharide such as starch, chitosan, chitin, agarose, agar, locust bean gum, carrageenan, gellan gum, pectin, alginate, xanthan, guar gum, and the like.

In some embodiments, the composition can include up to about 2% by weight of the final composition of sodium hyaluronate as the single gelling agent. In some embodiments, the composition can include more than about 4% or more than about 5% by weight of the final composition of gelatin as the single gelling agent. In some embodiments, the composition can include up to about 10% or up to about 8% starch as the single gelling agent. In some embodiments, the composition can include more than about 5% or more than about 10% by weight of the final composition of collagen as the gelling agent. In some embodiments, about 0.1% to about 10% or about 0.5% to about 3% by weight of the final composition of chitin can be used as the gelling agent. In some embodiments, from about 0.5% to about 5% by weight of the final composition of corn starch or from about 5% to about 10% by weight of the final composition of corn starch can be used as the gelling agent. In some embodiments, more than about 2.5 wt % by weight of the final composition of alginate can be used in the composition as the gelling agent. In some embodiments, the percentages by weight percent of the final composition of the gelling agents can be as follows: cellulose gel (from about 0.3% to about 2.0%), konjac gum (from about 0.5% to about 0.7%), carrageenan gum (from about 0.02% to about 2.0%), xanthan gum (from about 0.01% to about 2.0%), acacia gum (from about 3% to about 30%), agar (from about 0.04% to about 1.2%), guar gum (from about 0.1% to about 1%), locust bean gum (from about 0.15% to about 0.75%), pectin (from about 0.1% to about 0.6%), tara gum (from about 0.1% to about 1.0%), polyvinylypyrrolidone (from about 1% to about 5%), sodium polyacrylate (from about 1% to about 10%). Other gelling agents can be used in amounts sufficient to gel the composition or to sufficiently thicken the composition. It will be appreciated that lower amounts of the above gelling agents may be used in the presence of another gelling agent or a thickener.

The composition of the present disclosure may be further encapsulated, e.g., in a membrane. Such a membrane may be transparent, and/or substantially, or fully impermeable. The membrane may be impermeable to liquid but permeable to gases such as air. In some embodiments, the composition may form a membrane that encapsulates the chromophore(s) of the biophotonic topical composition, where the membrane may be substantially impermeable to liquid and/or gas. The membrane may be formed of one or more lipidic agents, polymers, gelatin, cellulose or cyclodextrins, or the like. In some embodiments, the membrane is translucent or transparent to allow light to infiltrate to and from the chromophore(s). In some embodiments, the composition is a dendrimer with an outer membrane comprising poly(propylene amine). In some embodiments, the outer membrane comprises gelatin.

### Polyols

According to some embodiments, the compositions of the methods and uses of the present disclosure may optionally further comprise one or more polyols. Suitable polyols that may be included in the composition include, but are not limited to a diol, a triol, a saccharide, glycerine, butane-1,2,3-triol, butane-1,2,4-triol, hexane-1,2,6-triol, propylene glycol, butanediol, butenediol, butynediol, pentanediol, hexanediol, octanediol, neopentyl glycol, 2-methyl-1,3-propanediol, diethylene glycol, triethylene glycol, tetraethylene glycol, dipropylene glycol and dibutylene glycol. In some embodiments when the composition of the disclosure includes one or more polyols, the polyol is glycerine. In some embodiments when the composition of the disclosure includes one or more polyols, the polyol is propylene glycol. In some embodiments when the composition of the disclosure includes one or more polyols, the polyol is a combination of glycerine and propylene glycol.

In some embodiments, one or more polyols are present in an amount of about 5-75% by weight of the total composition, such as 5-75% by weight of the total composition. In some embodiments, one or more polyols are present in an amount of about 10-75% by weight of the total composition, such as 10-75% by weight of the total composition. In some embodiments, one or more polyols are present in an amount of about 15-75% by weight of the total composition, such as 15-75% by weight of the total composition. In some embodiments, one or more polyols are present in an amount of about 20-75% by weight of the total composition, such as 20-75% by weight of the total composition.

### Additional Components

The compositions, methods, and uses of the disclosure can also include other ingredients such as humectants (e.g., glycerine, ethylene glycol, and propylene glycol), preservatives such as parabens, and pH adjusters such as sodium hydroxide, sodium bicarbonate, and HCl.

In some embodiments, the pH of the composition is in or adjusted to the range of about 4 to about 10. In some embodiments, the pH of the composition is in or adjusted to the range of about 4 to about 9. In some embodiments, the pH of the composition is in or adjusted to the range of about 4 to about 8. In some embodiments, the pH of the composition is within the range of about 4 to about 7. In some embodiments, the pH of the composition is within the range of about 4 to about 6.5. In some embodiments, the pH of the composition is within the range of about 4 to about 6. In some embodiments, the pH of the composition is within the range of about 4 to about 5.5. In some embodiments, the pH of the composition is within the range of about 4 to about 5. In some embodiments, the pH of the composition is within the range of about 5.0 to about 8.0. In some embodiments, the pH of the composition is within the range of about 6.0 to about 8.0. In some embodiments, the pH of the composition is within the range of about 6.5 to about 7.5. In some embodiments, the pH of the composition is within the range of about 5.5 to about 7.5.

In some embodiments, the pH of the composition is in or adjusted to the range of 4 to 10. In some embodiments, the pH of the composition is in or adjusted to the range of 4 to 9. In some embodiments, the pH of the composition is in or adjusted to the range of 4 to 8. In some embodiments, the pH of the composition is within the range of 4 to 7. In some embodiments, the pH of the composition is within the range of 4 to 6.5. In some embodiments, the pH of the composition is within the range of 4 to 6. In some embodiments, the pH of the composition is within the range of 4 to 5.5. In some embodiments, the pH of the composition is within the range of 4 to 5. In some embodiments, the pH of the composition is within the range of 5.0 to 8.0. In some embodiments, the pH of the composition is within the range of 6.0 to 8.0. In some embodiments, the pH of the composition is within the range of 6.5 to 7.5. In some embodiments, the pH of the composition is within the range of 5.5 to 7.5.

In some embodiments, the compositions of the disclosure also include an aqueous substance (water) or an alcohol. Alcohols include, but are not limited to, ethanol, propanol, isopropanol, butanol, iso-butanol, t-butanol or pentanol. In some embodiments, the chromophore or combination of chromophores is in solution in a medium of the composition. In some embodiments, the chromophore or combination of chromophores is in solution in a medium of the composition, wherein the medium is an aqueous substance.

### Methods of Use and Treatment

As discussed above, pain is a complex sensation which is mediated by the nervous system and is perceived as an unpleasant sensory and emotional experience. Pain can be subdivided into two broad classes, nociceptive pain or neuropathic pain. Nociceptive pain is the result of direct activation of sensory nerve receptors (called nociceptors) in the skin or soft tissue in response to tissue injury and its resulting inflammatory response. Nociceptive pain can be further subdivided according to the mode of noxious stimulation or stimulus (i.e. mechanical, thermal, and chemical). However, activation of nociceptors is not required for the sensation of pain. Neuropathic pain is the result of damage or disease to any part of the central or peripheral nervous system. The damaged, dysfunctional, or injured nerve fibers become unusually sensitive and develop spontaneous pathological activity which results in a burning or electric sensation of pain.

Pain can also be subdivided into acute and chronic pain. Acute pain, for the most part, results from disease, inflammation, or injury to tissues. This type of pain generally comes on suddenly, for example, after trauma or surgery. In some instances, it can become chronic. Chronic pain is widely believed to represent disease itself. Chronic pain persists over a longer period of time than acute pain and is resistant to most medical treatments. It can, and often does, cause severe problems for patients.

### Methods of Scoring Pain

Various methods of rating or scoring pain levels exist in the literature. Due to the subjective nature of pain, the majority of pain assessment methods are based on either patients or physician's assessment of pain severity. For example, pain can be scored using a numerical analog score, a verbal assessment score, a verbal pain score, a visual analog score, etc. Such scoring systems are typically based on a continuum of possible scores or a series of possible scores where the lowest possible score is no pain, and the highest possible score is the worst pain imaginable. Other such scoring systems can be based on a scale representing low, medium, or high levels of pain.

One commonly used scoring system is a verbal pain assessment method where the patient is asked to verbally assign a score to their pain and/or discomfort at several points during the treatment. The score can be based on a 10 point scale, where 0 represents no pain and 10 represents the worst pain imaginable. In one example, when such a 10 point pain scale is used, a score of 8 or below can be considered to be a desirable level of pain for a patient to experience during or following a dermatological treatment. A reduction in an average pain score of one point on this scale can be considered to be a desirable and effective reduction in pain. For example, if for a patient a pain-reducing method results in a reduction in an average pain score of at least one point on this scale, the method can be considered to have been effective in reducing the patient's pain. If for a patient a pain-reducing method results in a reduction in an average pain score of at least two points on this scale, the method can be considered to have been more effective in reducing the patient's pain.

Another commonly used scoring system is the visual analogue scale or visual analog scale (VAS). The VAS is a psychometric response scale which can be used in questionnaires or hospital settings. It is a measurement instrument for subjective characteristics or attitudes that cannot be directly measured. For example, the amount of pain that a patient feels ranges across a continuum from none to an extreme amount of pain. From the patient's perspective, this spectrum appears continuous because their pain does not take discrete jumps, as a categorization of none, mild, moderate and severe would suggest. It was to capture this idea of an underlying continuum that the VAS was devised. The VAS for pain often consists of a 10 cm line with two end-points representing ‘no pain’ (score of 0) and ‘pain as bad as it could possibly be’ (score of 10). Patients are asked to rate their pain by placing a mark on the line corresponding to their current level of pain. The distance along the line from the ‘no pain’ (score of 0) marker is then measured with a ruler giving a pain score out of 10. A reduction in an average pain score of one point on this scale can be considered to be a desirable and effective reduction in pain. For example, if for a patient a pain-reducing method results in a reduction in an average pain score of at least one point on this scale, the method can be considered to have been effective in reducing the patient's pain. If for a patient a pain-reducing method results in a reduction in an average pain score of at least two points on this scale, the method can be considered to have been more effective in reducing the patient's pain.

The compositions suitable for use in the methods of the present disclosure may be selected from any of the embodiments of the compositions described above. For instance, the compositions useful in the method of the present disclosure may comprise a chromophore that undergoes at least partial photobleaching upon application of light.

The chromophore may absorb at a wavelength of from about 200 nm to about 800 nm, from about 200 nm to about 700 nm, from about 200 nm to about 600 nm or from about 200 nm to about 500 nm. In some embodiments, the chromophore absorbs at a wavelength of from about 200 nm to about 600 nm. In some embodiments, the chromophore absorbs light at a wavelength of from about 200 nm to about 300 nm, from about 250 nm to about 350 nm, from about 300 nm to about 400 nm, from about 350 nm to about 450 nm, from about 400 nm to about 500 nm, from about 450 nm to about 650 nm, from about 600 nm to about 700 nm, from about 650 nm to about 750 nm or from about 700 nm to about 800 nm. In some embodiments, suitable compositions for the methods of the present disclosure may further comprise at least one additional chromophore (e.g., a second chromophore). The absorption spectrum of the second chromophore overlaps at least about 80%, about 70%, about 60%, about 50%, about 40%, about 30%, or about 20% with the emission spectrum of the first chromophore. In some embodiments, the first chromophore has an emission spectrum that overlaps at least 1-10%, 5-15%, 10-20%, 15-25%, 20-30%, 25-35%, 30-40%, 35-45%, 50-60%, 55-65% or 60-70% with an absorption spectrum of the second chromophore.

In the methods of the present disclosure, any source of actinic light can be used to illuminate the compositions. Any type of halogen, LED or plasma arc lamp or laser may be suitable. The primary characteristic of suitable sources of actinic light will be that they emit light in a wavelength (or wavelengths) appropriate for activating the one or more photoactivators present in the composition. In some embodiments, an argon laser is used. In some embodiments, a potassium-titanyl phosphate (KTP) laser (e.g., a GreenLight™ laser) is used. In another embodiment, sunlight may be used. In some embodiments, a LED photocuring device is the source of the actinic light. In some embodiments, the source of the actinic light is a source of light having a wavelength between about 200 nm to about 800 nm. In some embodiments, the source of the actinic light is a source of visible light having a wavelength between about 400 nm and about 700 nm. In some embodiments, the source of the actinic light is a source of visible light having a wavelength between about 400 nm and about 600 nm. In some embodiments, the source of the actinic light is a source of visible light having a wavelength between about 400 nm and about 550 nm. In some embodiments, the source of the actinic light is a source of visible light having a wavelength between about 380 nm and about 700 nm. In some embodiments, the source of the actinic light is a source of visible light having a wavelength between about 380 nm and about 600 nm. In some embodiments, the source of the actinic light is a source of visible light having a wavelength between about 380 nm and about 550 nm. In some embodiments, the source of the actinic light is a source of light having a wavelength between 200 nm to 800 nm. In some embodiments, the source of the actinic light is a source of visible light having a wavelength between 400 nm and 700 nm. In some embodiments, the source of the actinic light is a source of visible light having a wavelength between 400 nm and 600 nm. In some embodiments, the source of the actinic light is a source of visible light having a wavelength between 400 nm and 550 nm. In some embodiments, the source of the actinic light is a source of visible light having a wavelength between 380 nm and 700 nm. In some embodiments, the source of the actinic light is a source of visible light having a wavelength between 380 nm and 600 nm. In some embodiments, the source of the actinic light is a source of visible light having a wavelength between 380 nm and 550 nm. In some embodiments, the composition of the disclosure is illuminated with violet and/or blue light. Furthermore, the source of actinic light should have a suitable power density. Suitable power density for non-collimated light sources (LED, halogen or plasma lamps) are in the range from about 1 mW/cm² to about 200 mW/cm². Suitable power density for laser light sources is in the range from about 0.5 mW/cm² to about 0.8 mW/cm².

In some embodiments, the light source is a LED light. In some embodiments of the foregoing and following, the LED light is a source of light having a wavelength between 500 nm and 700 nm, such as between 620 nm and 640 nm. In certain such embodiments, the source of light has a minimal peak power density between 600-800 μW, e.g., 719 μW.

In some embodiments, the LED light is a source of light having a wavelength between 400 nm and 500 nm, such as between 425 nm and 450 nm. In certain such embodiments, the source of light has a minimal peak power density between 700-900 μW, such as 838 μW. In some embodiments of the foregoing and following, the source of light has an average minimal peak power density between 300-400 μW over 30 doses of working time, wherein each dose comprises about 10 minutes of illumination.

In some embodiments of the methods of the present disclosure, the light has an energy at the subject's skin of between about 1 mW/cm² and about 500 mW/cm², about 1-300 mW/cm², or about 1-200 mW/cm², wherein the energy applied depends at least on the condition being treated, the wavelength of the light, the distance of the subject's skin from the light source, and the thickness of the composition. In some embodiments, the light at the subject's skin is between about 1-40 mW/cm², or about 20-60 mW/cm², or about 40-80 mW/cm², or about 60-100 mW/cm², or about 80-120 mW/cm², or about 100-140 mW/cm², or about 120-160 mW/cm², or about 140-180 mW/cm², or about 160-200 mW/cm², or about 110-240 mW/cm², or about 110-150 mW/cm², or about 190-240 mW/cm².

In some embodiments, a mobile device can be used to activate embodiments of the composition of the present disclosure, wherein the mobile device can emit light having an emission spectrum which overlaps an absorption spectrum of the chromophore in the composition. The mobile device can have a display screen through which the light is emitted and/or the mobile device can emit light from a flashlight which photoactivates the composition.

In some embodiments, a display screen on a television or a computer monitor can be used to activate the composition, wherein the display screen can emit light having an emission spectrum which overlaps an absorption spectrum of a photoactive agent in the photoactivatable composition.

In some embodiments, the chromophore or combination of chromophores can be photoactivated by ambient light which may originate from the sun or other light sources. Ambient light can be considered to be a general illumination that comes from all directions in a room that has no visible source. In some embodiments, the chromophore or combination of chromophores can be photoactivated by light in the visible range of the electromagnetic spectrum. Exposure times to ambient light may be longer than that to direct light.

In some embodiments, different sources of light can be used to activate the compositions, such as a combination of ambient light and direct LED light.

The duration of the exposure to actinic light required will be dependent on the surface of the treated area, the severity of the condition that is being treated, the power density, wavelength and bandwidth of the light source, the thickness of the composition, and the treatment distance from the light source. The illumination of the treated area by fluorescence may take place within seconds or even fragment of seconds, but a prolonged exposure period is beneficial to exploit the synergistic effects of the absorbed, reflected and reemitted light on the composition of the present disclosure and its interaction with the tissue being treated. In some embodiments, the time of exposure to actinic light of the tissue or skin or area of pain on which the composition has been applied is a period between 1 second and 30 minutes. In some embodiments, the time of exposure to actinic light of the tissue or skin or area of pain on which the composition has been applied is a period between 1 minute and 30 minutes. In some embodiments, the time of exposure to actinic light of the tissue, skin or area of pain on which the composition has been applied is a period between 1 minute and 5 minutes. In some embodiments, the time of exposure to actinic light of the tissue, skin or area of pain on which the composition has been applied is a period between 1 minute and 5 minutes. In another embodiment, the time of exposure is from about 20 seconds to about 5 minutes, or from between about 60 seconds and about 5 minutes. In another embodiment, the time of exposure to actinic light of the tissue on which the composition has been applied is a period of less than about 5 minutes. In another embodiment, the time of exposure is between about 20 seconds to about 5 minutes, or between about 60 seconds and about 5 minutes per cm² of the area to be treated, so that the total time of exposure of a 10 cm² area would be between 10 minutes and 50 minutes.

In some embodiments, the composition is illuminated for a period between 1 minute and 3 minutes. In some embodiments, light is applied for a period of 1-30 seconds, 1-60 seconds, 15-45 seconds, 30-60 seconds, 0.75-1.5 minutes, 1-2 minutes, 1.5-2.5 minutes, 2-3 minutes, 2.5-3.5 minutes, 3-4 minutes, 3.5-4.5 minutes, 4-5 minutes, 5-10 minutes, 10-15 minutes, 15-20 minutes, 20-25 minutes, or 20-30 minutes. In some embodiments, light is applied for a period of 1 second. In some embodiments, light is applied for a period of 5 seconds. In some embodiments, light is applied for a period of 10 seconds. In some embodiments, light is applied for a period of 20 seconds. In some embodiments, light is applied for a period of 30 seconds. In some embodiments, the composition is illuminated for a period less than 30 minutes. In some embodiments, the composition is illuminated for a period less than 20 minutes. In some embodiments, the composition is illuminated for a period less than 15 minutes. In some embodiments, the composition is illuminated for a period less than 10 minutes. In some embodiments, the composition is illuminated for a period less than 5 minutes. In some embodiments, the composition is illuminated for a period less than 1 minute. In some embodiments, the composition is illuminated for a period less than 30 seconds. In some embodiments, the composition is illuminated for a period less than 20 seconds. In some embodiments, the composition is illuminated for a period less than 10 seconds. In some embodiments, the composition is illuminated for a period less than 5 seconds. In some embodiments, the composition is illuminated for a period less than 1 second. In some embodiments, the source of actinic light is in continuous motion over the treated area for the appropriate time of exposure. In some embodiments, multiple applications of the composition and actinic light are performed. In some embodiments, the tissue, skin or area of pain is exposed to actinic light at least two, three, four, five or six times. Also, the entire treatment may be repeated in its entirety as may be required by the patient. In some embodiments, a fresh application of the composition is applied before exposure to actinic light.

In the methods of the present disclosure, the composition may be optionally removed from the site of treatment following application of light. In some embodiments, the composition is left on the treatment site for more than 30 minutes, more than one hour, more than 2 hours, or more than 3 hours. It can be illuminated with ambient light. To prevent drying, the composition can be covered with a transparent or translucent cover such as a polymer film, or an opaque cover which can be removed before illumination.

The compositions of the disclosure may be applied at regular intervals such as once a week, or at an interval deemed appropriate by the physician or veterinarian. In some embodiments, the compositions of the disclosure are applied once per week for one week. In some embodiments, the compositions of the disclosure are applied once per week for two weeks. In some embodiments, the compositions of the disclosure are applied once per week for three weeks. In some embodiments, the compositions of the disclosure are applied once per week for four weeks. In some embodiments, the compositions of the disclosure are applied once per week for five weeks. In some embodiments, the compositions of the disclosure are applied once per week for six weeks. In some embodiments, the compositions of the disclosure are applied once per week for seven weeks. In some embodiments, the compositions of the disclosure are applied once per week for eight or more weeks.

In some embodiments, the compositions of the disclosure are applied twice per week for one week. In some embodiments, the compositions of the disclosure are applied twice per week for two weeks. In some embodiments, the compositions of the disclosure are applied twice per week for three weeks. In some embodiments, the compositions of the disclosure are applied twice per week for four weeks. In some embodiments, the compositions of the disclosure are applied twice per week for five weeks. In some embodiments, the compositions of the disclosure are applied twice per week for six weeks. In some embodiments, the compositions of the disclosure are applied twice per week for seven weeks. In some embodiments, the compositions of the disclosure are applied twice per week for eight or more weeks.

In some embodiments, the compositions of the disclosure are applied three times or more per week for one week. In some embodiments, the compositions of the disclosure are applied three times or more per week for two weeks. In some embodiments, the compositions of the disclosure are applied three times or more per week for three weeks. In some embodiments, the compositions of the disclosure are applied three times or more per week for four weeks. In some embodiments, the compositions of the disclosure are applied three times or more per week for five weeks. In some embodiments, the compositions of the disclosure are applied three times or more per week for six weeks. In some embodiments, the compositions of the disclosure are applied three times or more per week for seven weeks. In some embodiments, the compositions of the disclosure are applied three times or more per week for eight or more weeks.

For any of the methods described herein, the embodiments of this disclosure contemplate the use of any of the compositions, or mixtures of them, described throughout the application. In addition, in various embodiments of any of the methods described herein, combinations of any step or steps of one method with any step or steps from another method may be employed.

### Reducing Pain

The compositions of the present disclosure are useful in the treatment or reduction of pain. In certain embodiments, the pain may be nociceptive pain or neuropathic pain. In certain such embodiments, the pain is acute pain or chronic pain. The compositions of the present disclosure are useful in the treatment or reduction of pain due to the localized, non-systemic application directly to the area of pain. In certain such embodiments, the pain does not originate from a stimulus in an orofacial region. In certain such embodiments, the reduction of pain is not dependent on the treatment of the underlying medical condition.

Pain that may be treated or reduced by the biophotonic compositions and methods of the present disclosure include, for example, pain from chronic wounds initiated in different ways (e.g., venous leg ulcers and diabetic foot ulcers) and with varying characteristics. In some embodiments, the present disclosure provides biophotonic compositions and methods for treating and/or reducing the pain associated with, for example, widespread pain, localized pain, nociceptive pain, inflammatory pain, peripheral neuropathic pain, peripheral neurogenic pain, peripheral neuralgia, low back pain, postoperative pain, visceral pain, and pelvic pain; allodynia; Anesthesia dolorosa; causalgia; dysesthesia; fibromyalgia; hyperalgesia; hyperesthesia; ischemic pain; sciatic pain; pain associated with cystitis including, but not limited to, interstitial cystitis; pain associated with multiple sclerosis; pain associated with arthritis; pain associated with osteoarthritis; pain associated with rheumatoid arthritis; pain associated with chronic wounds; pain associated with burns; and pain associated with cancer. Pain that may be treated by the biophotonic compositions and methods of the present disclosure also include pain associated with acute wounds such as pain associated with burns or pain associated with postoperative care.

The compositions of the disclosure may decrease pain within various periods of initiating biophotonic therapy. In some embodiments, the compositions of the disclosure decrease pain within two to four weeks of initiating biophotonic therapy. In some embodiments, the compositions of the disclosure decrease pain within one to two weeks of initiating biophotonic therapy. In some embodiments, the compositions of the disclosure decrease pain in less than one week of initiating biophotonic therapy.

The biophotonic compositions disclosed herein can be used as a first line of treatment, or a second line of treatment, as shown herein. Whether used as a first line of treatment or a second line of treatment, the biophotonic compositions disclosed herein can be used in combination with other known standard treatment regime (e.g., for wound care), as further described below.

### Combination Therapies

Any of the compositions, methods, or uses of this disclosure are useful in combination with other therapeutics.

In some embodiments, the phrase “combination therapy” embraces the administration of the any of the compositions described herein, and an additional therapeutic agent, or mixtures of them, as part of a specific treatment regimen intended to provide a beneficial effect from the co-action of these therapeutic agents. Administration of these therapeutic agents in combination typically is carried out over a defined time period (usually minutes, hours, days or weeks depending upon the combination selected). “Combination therapy” is intended to embrace administration of these therapeutic agents in a sequential manner, that is, wherein each therapeutic agent is administered at a different time, as well as administration of these therapeutic agents, or at least two of the therapeutic agents, in a substantially simultaneous manner. The therapeutic agents can be administered by the same route or by different routes. For example, a first therapeutic agent of the combination selected may be administered by intravenous injection or orally while the composition of the disclosure is administered topically. Alternatively, for example, all therapeutic agents may be administered topically. The sequence in which the therapeutic agents are administered is not narrowly critical. “Combination therapy” also can embrace the administration of the compositions as described herein in further combination with other biologically active ingredients (such as, but not limited to, a second and different therapeutic agent) and non-drug therapies (such as, but not limited to, surgery or radiation).

In some embodiments, the therapeutic agents administered in combination therapy simultaneously, separately, or sequentially with any of the compounds and compositions of this disclosure, or mixtures thereof, can comprise, but are not limited to: a non-steroidal anti-inflammatory drug (NSAID), an anti-inflammatory agent, a corticosteroid, an anti-allergic agent, a steroid drug, one or more of the antimicrobial agents described above, one or more collagens and/or agents that promote collagen synthesis described above, or mixtures thereof.

In some embodiments, any of the compositions described herein can allow the combination therapeutic agents and/or compositions described herein or mixtures thereof to be administered at a low dose, that is, at a dose lower than has been conventionally used in clinical situations.

Alternatively, the methods and combinations of this disclosure maximize the therapeutic effect at higher doses.

In some embodiments, when administered as a combination, the therapeutic agents can be formulated as separate compositions which are given at the same time or different times, or the therapeutic agents can be given as a single composition.

The methods as disclosed herein can be used alone or in combination with other methods of reducing or relieving pain. For example, these methods can be used in conjunction with topical analgesics, topical anesthetics, injectable anesthetics, etc. The methods as disclosed herein can be used to reduce the dosage of an analgesic and/or anesthetic required to relieve and/or reduce the level of pain experienced by a patient experiencing a pain-inducing medical condition.

A number of different types of topical anesthetics at various concentrations and dosages are commonly used to reduce or relieve the pain of medical conditions. For example, commonly used topical anesthetics include of lidocaine, prilocaine, tetracaine, benzocaine, dibucaine, oxybuprocaine, pramoxine, proparacaine, proxymetacaine, as well as combinations of topical anesthetics and other active ingredients. The non-pharmacologic methods as disclosed herein can be used to augment the effect of these active ingredients in order to relieve or reduce pain.

For example, the methods as disclosed herein can be used to reduce the concentration and/or dosage of an active ingredient that is required to produce a desired level of pain relief and/or reduction. If combining a pain-reducing method as disclosed herein with an active ingredient produces a reduction in an average pain score of at least one point on a ten point pain scale, the method can be considered to have been effective in relieving and/or reducing the patient's pain. If combining a pain-reducing method as disclosed herein with an active ingredient produces a reduction in an average pain score of at least two points on a ten point pain scale, the method can be considered to have been more effective in relieving and/or reducing the patient's pain. Alternatively, if combining a pain-reducing method as disclosed herein with an active ingredient can reduce the dosage and/or concentration of the active ingredient required by at least 10% in order to produce a similar average pain scale as compared to the use of the active ingredient alone, the method can be considered to have been effective in reducing the dosage and/or concentration of active ingredient required. If combining a pain-reducing method as disclosed herein with an active ingredient can reduce the dosage and/or concentration of the active ingredient required by at least 20% in order to produce a similar average pain scale as compared to the use of the active ingredient alone, the method can be considered to have been more effective in reducing the dosage and/or concentration of active ingredient required.

### Kits

The present disclosure also provides kits for preparing and/or applying any of the compositions of the present disclosure for reducing pain associated with a medical condition in a subject. The kit may include a composition as described above (e.g., a biophotonic topical composition), and may also include a light source, an apparatus for applying or removing the composition, and instructions of use for the composition and/or a light source. In some embodiments, the composition comprises at least one oxidant and at least one chromophore capable of activating the oxidant. In other embodiments, the composition comprises at least one chromophore and at least one salt selected from bicarbonate salts, carbonate salts, or a combination of foregoing salts.

In some embodiments, the kit includes more than one composition, for example, a first and a second composition. The first composition may include at least one oxidant and the second composition may include at least one chromophore capable of activating the at least one oxidant in the first composition. In certain such embodiments, the oxidant is chosen from hydrogen peroxide, carbamide peroxide and benzoyl peroxide. In certain such embodiments, the first and/or second composition further comprises one or more healing agents. In certain such embodiments, the first and/or second composition further comprises one or more gelling agents.

In some embodiments, the kit includes more than one composition, for example, a first and a second composition. The first composition may include at least one chromophore and the second composition may include at least one salt selected from bicarbonate salts, carbonate salts, or a combination of foregoing salts. In certain such embodiments, the first and/or second composition further comprises one or more healing agents. In certain such embodiments, the first and/or second composition further comprises one or more gelling agents.

In some embodiments, the first composition may comprise the at least one oxidant, and the second composition may comprise at least one chromophore capable of activating the at least one oxidant in a liquid or as a powder. In certain such embodiments, the oxidant is chosen from hydrogen peroxide, carbamide peroxide and benzoyl peroxide. In certain such embodiments, the first and/or second composition further comprises one or more healing agents. In certain such embodiments, the first and/or second composition further comprises one or more gelling agents.

In some embodiments, the first composition may comprise the at least one chromophore, and the second composition may comprise at least one salt selected from bicarbonate salts, carbonate salts, or a combination of foregoing salts. In certain such embodiments, the first and/or second composition further comprises one or more healing agents. In certain such embodiments, the first and/or second composition further comprises one or more gelling agents.

In some embodiments, the kit includes containers comprising the compositions of the present disclosure. In some embodiments, the kit includes a first container comprising at least one oxidant, and a second container comprising the at least one chromophore capable of activating the oxidant. In certain such embodiments, the oxidant is chosen from hydrogen peroxide, carbamide peroxide and benzoyl peroxide. In certain such embodiments, the first and/or second composition further comprises one or more healing agents. In certain such embodiments, the first and/or second composition further comprises one or more gelling agents.

In some embodiments, the kit includes containers comprising the compositions of the present disclosure. In some embodiments, the kit includes a first container comprising the at least one chromophore capable of activating the oxidant, and a second container comprising at least one salt selected from bicarbonate salts, carbonate salts, or a combination of foregoing salts. In certain such embodiments, the first and/or second composition further comprises one or more healing agents. In certain such embodiments, the first and/or second composition further comprises one or more gelling agents.

The containers may be light impermeable, air-tight and/or leak resistant. Exemplary containers include, but are not limited to, syringes, vials, or pouches. The first and second compositions may be included within the same container but separated from one another until a user mixes the compositions. In some embodiments, the container may be a dual-chamber syringe where the contents of the chambers mix on expulsion of the compositions from the chambers. In some embodiments, the pouch may include two chambers separated by a frangible membrane. In some embodiments, one component may be contained in a syringe and injectable into a container comprising the second component.

The composition may also be provided in a container comprising one or more chambers for holding one or more components of the composition, and an outlet in communication with the one or more chambers for discharging the composition from the container.

In some embodiments, the kit comprises a systemic or topical drug for augmenting the treatment of the composition. For example, in certain such embodiments, the kit may include a systemic or topical agent, e.g., an anesthetics or anti-inflammation agent, for reducing pain. Written instructions on how to use the composition for pain reduction in accordance with the present disclosure may be included in the kit, or may be included on or associated with the containers comprising the compositions of the present disclosure.

In some embodiments, the kit may comprise a further component which is a dressing. The dressing may be a porous or semi-porous structure for receiving the composition. The dressing may comprise woven or non-woven fibrous materials.

In some embodiments of the kit, the kit may further comprise a light source such as a portable light with a wavelength appropriate to activate the chromophore in the composition. The portable light may be battery operated or re-chargeable. In some embodiments, the light source is an actinic light source.

In some embodiments, the kit may further comprise one or more waveguides.

Identification of equivalent compositions, methods and kits are well within the skill of the ordinary practitioner and would require no more than routine experimentation, in light of the teachings of the present disclosure. Practice of the disclosure will be still more fully understood from the following examples, which are presented herein for illustration only and should not be construed as limiting the disclosure in any way.

### EXAMPLES

The examples below are given so as to illustrate the practice of various embodiments of the present disclosure. They are not intended to limit or define the entire scope of this disclosure.

It should be appreciated that the disclosure is not limited to the particular embodiments described and illustrated herein but includes all modifications and variations falling within the scope of the disclosure as defined in the appended embodiments.

### Example 1—Chronic Wounds

The study was designed to recruit a total of 100 patients at 12 clinical sites, but only 99 patients were included in total in the final analysis. As noted herein, reference is sometimes made to an “interim study” which includes an initial subset of 33 patients for which data were initially available. In certain instances, data from the interim study are disclosed herein.

An initial screen visit to identify eligible patients was conducted prior to beginning treatment. The recruited patients would be participating for their treatment of chronic wounds (pressure ulcers (PUs), venous leg ulcers (VLUs) and diabetic foot ulcers (DFUs) in a real-life context clinical setting using a biophotonic composition of the present disclosure comprising a synthetic chromophore (Eosin Y) and an oxidant in a carbopol-based gel. For this study, the treatment period was designed to be two applications of the biophotonic composition per week over a period of treatment of 16 weeks for both PUs and VLUs, and over a period of 24 weeks for DFUs. A follow-up period of 8-week, commencing upon the termination of treatment, was used to confirm the persistence of wound closure once a wound has closed.

Patient's ulcer was first cleansed with normal saline. If needed, sharp debridement was performed, to remove excess necrotic tissue or foreign materials. The biophotonic composition of the present disclosure was then prepared, and an approximately 2 mm thick layer of the composition was applied on the surface of the ulcer. The patient was then supplied with eye protectors and the persons administering the treatment were requested to wear goggles, to protect their eyes. The KLOX Multi-LED light (THERA™ lamp) delivering non-coherent blue light in a range 400-470 nm was then placed over the wound area, at a measured distance of 5 cm, using the attached lamp measuring probe. The ulcer was illuminated for a period of 5 minutes. Once the illumination period was completed, the composition was removed from the wound surface with a sterile spatula. The wound was then wiped with a moist towel or gauze, and then irrigated with saline solution. A non-adherent dressing was applied, either self-fixating or fixed to the patient with tape and/or bandages, to prevent any contact between the wound and the external environment. Local standard of care was then followed.

Efficacy was assessed using the following endpoints:
Rate of complete wound closure (wound closure being defined as skin re-epithelialization without drainage or dressing requirements confirmed at two consecutive visits, two weeks apart);
Rate of complete wound closure by week 16 for PUs and VLUs, and by week 24 for DFUs;
Time to complete wound closure;
Wound area reduction over time;
Wound volume reduction over time;
Incidence of wound breakdown, following closure;
Impact of treatment on patients' quality of life; and
Ease of use by healthcare professionals.

From the one hundred screened patients who enrolled in the study, a total of ninety-nine were enrolled and treated at least once. Of the thirty-three patients in the interim study, sixteen patients completed the study period as per protocol, while seventeen patients who met the criteria were enrolled and treated, but discontinued early. Among the seventeen patients who discontinued from the study early, eleven were being treated for diabetic foot ulcers (DFUs), two were treated for pressure ulcers (PUs) and four were treated for venous leg ulcers (VLUs).

With respect to the demographic and other baseline criteria for the ninety-nine patients of the trial, the mean age of the patients was 68.70 years, with the youngest patient being 38 years-old whereas the oldest patient was 88 years-old. The average age was lower in the PU patient (60.18 years) and DFU patient (69.27 years) groups compared to the patients in the VLU group (70.80 years). Regarding the gender of the thirty-three patients, as shown in Table 1 below, two-thirds of the patients were males and one-third were females.

TABLE 1
Patients gender-all wounds

| Gender | Number of patients | % |
|---|---|---|
| Male | 67 | 67.7 |
| Female | 32 | 32.3 |
| Total | 99 | 100 |

As can be seen in Table 2 below, each chronic wound type had representation in both male and female populations for the ninety-nine patients presented in the final analysis.

TABLE 2
Patients gender-number of patients by type of wound

| Gender | PU | DFU | VLU |
|---|---|---|---|
| Male | 13 | 25 | 29 |
| Female | 2 | 7 | 23 |
| Total | 15 | 32 | 52 |

Other demographic information for the ninety-nine patients included ethnicity (all ninety-nine were Caucasian), smoking status as of the date of screening (fifty-four never smoked, thirty-eight had stopped smoking, and seven patients were smokers), vital signs including heart rate, blood pressure, body temperature and respiration rate (none of the ninety-nine patients vital signs was considered as clinically significant by the investigators) and body-mass index.

Among the three different types of chronic ulcers treated in the 99 patients included in the final analysis, staging was assessed by the investigators at the patient's screening visit for PUs and DFUs as shown below in Tables 3 and 4, respectively.

The classification used for Pressure ulcers was the staging system proposed by the National Pressure Ulcer Advisory Panel (NPUAP) and the European Pressure Ulcer Advisory Panel (EPUAP). (Source: National Pressure Ulcer Advisory Panel and European Pressure Ulcer Advisory Panel. Pressure Ulcer Treatment. Quick Reference Guide 2009. Available at: http://www.npuap.org). Only Stages II and III Pressure ulcers were accepted in the study. As shown on Table 6 (below), the majority (60%) of the PUs enrolled were classified as Stage III at study entry.

TABLE 3
EPUAP/NPUAP Staging-Pressure Ulcers

| Stage | Description | Additional Description |
|---|---|---|
| STAGE | Intact skin with nonblanchable | The area may be painful, firm, |
| I | redness of a localized area | soft, warmer or cooler as |
| | usually over a bony prominence, | compared to adjacent tissue. |
| | Darkly pigmented skin may not | Stage I may be difficult to |
| | have visible blanching; its color | detect in individuals with dark |
| | may differ from the surrounding | skin tones. May indicate “at |
| | area. | risk” persons (a heralding sign |
| | | of risk). |
| STAGE | Partial thickness loss of dermis | Presents as a shiny or dry |
| II | presenting as a shallow open | shallow ulcer without slough |
| | ulcer with a red pink wound bed, | or bruising*. This stage |
| | without slough. May also present | should not be used to describe |
| | as an intact or open/ruptured | skin tears, tape burns, |
| | serum-filled blister. | perineal dermatitis, |
| | | maceration or excoriation. |
| STAGE | Full thickness tissue loss. | The depth of a Stage III |
| III | Subcutaneous fat may be visible | pressure ulcer varies by |
| | but bone, tendon or muscle are | anatomical location. The bridge |
| | not exposed. Slough may be | of the nose, ear, occiput and |
| | present but does not obscure the | malleolus do not have |
| | depth of tissue loss. May include | subcutaneous tissue and Stage |
| | undermining and tunneling. | III ulcers can be shallow. In |
| | | contrast, areas of significant |
| | | adiposity can |
| | | develop extremely |
| | | deep Stage III pressure ulcers. |
| | | Bone/tendon is not visible or |
| | | directly palpable. |
| STAGE | Full thickness tissue loss with | The depth of a Stage IV |
| IV | exposed bone, tendon or muscle, | pressure ulcer varies by |
| | Slough or eschar may be present | anatomical location. The bridge |
| | on some parts of the wound bed. | of the nose, ear, occiput and |
| | Often include undermining and | malleolus do not have |
| | tunneling. | subcutaneous tissue and these |
| | | ulcers can be shallow. Stage IV |
| | | ulcers can extend into muscle |
| | | and/or supporting structures |
| | | (e.g., fascia, tendon or joint |
| | | capsule) making osteomyelitis |
| | | possible. Exposed bone/tendon |
| | | is visible or directly palpable. |
| UN- | Full thickness tissue loss in | Until enough slough and/or |
| STAGE- | which the base of the ulcer is | eschar is removed to expose |
| ABLE | covered by slough (yellow, tan, | the base of the wound, the true |
| | gray, green or brown) and/or | depth, and therefore stage, |
| | eschar (tan, brown or black) in | cannot be determined. Stable |
| | the wound bed. | (dry, adherent, intact without |
| | | erythema or fluctuance) eschar |
| | | on the heels serves as “the |
| | | body's natural (biological) |
| | | cover” and should not be |
| | | removed. |

*Bruising indicates suspected deep tissue injury.

The classification used for DFUs was the University of Texas classification. The University of Texas system assesses ulcer depth, the presence of wound infection, and the presence of clinical signs of lower-extremity ischemia. This system uses a matrix of grade on the horizontal axis and stage on the vertical axis. Only Diabetic foot ulcers with Stages 1A (superficial, non-infected, non-ischemic wound not involving tendon, capsules, or bone) or 2A (non-infected, non-ischemic wound penetrating to tendon or capsule but not in the bone or joint) were accepted. As shown on Table 7 (below), the majority (56.3%) of the DFUs enrolled were of Stage 2A.

TABLE 4
University of Texas Staging-Diabetic Foot Ulcers

| | 0 | 1 | 2 | 3 |
|---|---|---|---|---|
| A | Pre- or post- | Superficial | Wound | Wound |
| | ulcerative site | wound not | penetrating to | penetrating |
| | that has healed | involving | tendon or | bone or joint |
| | | tendon, capsule, | capsule | |
| | | or bone | | |
| B | With infection | With infection | With infection | With |
| | | | | infection |
| C | With ischemia | With ischemia | With ischemia | With |
| | | | | ischemia |
| D | With infection | With infection | With infection | With |
| | and ischemia | and ischemia | and ischemia | infection and |
| | | | | ischemia |

No classification was used for VLUs due to the lack of standardized staging which is internationally recognized.

For the ninety-nine patients presented in the study, Table 5 presents a breakdown with respect to chronic wound type of the patients; the majority of the patients were of either the DFU or VLU groups.

TABLE 5
Type of wounds included in the final analysis

| | Number of | Percentage of |
|---|---|---|
| Wound Type | patients | patients |
| DFU | 32 | 33.3% |
| PU | 15 | 15.2% |
| VLU | 52 | 52.5% |
| Total Number of | 99 | 100% |
| Patients | | |

The staging of the wounds for the PU and DFU patients presented in the final analysis are provided in Tables 6 and 7, respectively.

TABLE 6
Pressure Ulcers' Staging at Screening

| | Number of | |
|---|---|---|
| Stage | patients | % |
| II | 6 | 40.0 |
| III | 9 | 60.0 |
| Total | 15 | 100 |

TABLE 7
Diabetic Foot Ulcers' Staging at Screening

| | Number of | |
|---|---|---|
| Stage | patients | % |
| 1A | 14 | 43.8 |
| 2A | 18 | 56.3 |
| Total | 32 | 100 |

Given that the study was an observational study in real-life conditions, age of the wounds was not part of the eligibility criteria thereby explaining degree of variation from one wound to another when comparing patient-to-patient for the chronic wound type.

The average duration of the chronic ulcers (PUs, DFUs and VLUs combined) at screening for the study was 35.5 months. The average chronic ulcers duration was 64.1 months for PUs, 424.9 months for VLUs and 10.2 months for DFUs. The youngest ulcer had happened just prior to the patient's enrollment (PU) whereas the oldest ulcer was approximately 52 years old (VLU). Table 8 below presents a summary of all wounds combined, while Table 9 presents a summary specifically for the PU patients, Table 10 presents a summary for the DFU patients, and Table 11 presents a summary for the VLU patients.

TABLE 8
Chronic ulcers duration at screening
(including PUs, DFUs, and VLUs)

| Average | S.D. | Minimum | Maximum |
|---|---|---|---|
| (months) | (months) | (months) | (months) |
| 35.5 | 92.8 | 0.0 | 625.3 |

Note:
N = 99.
Duration calculated from date of wound diagnosis to date of screening, as reported by patients.
S.D.: Standard Deviation

TABLE 9
Pressure ulcers duration at screening

| Average | S.D. | Minimum | Maximum |
|---|---|---|---|
| (months) | (months) | (months) | (months) |
| 64.1 | 158.5 | 0.0 | 625.3 |

Note:
N = 15.
Duration calculated from date of wound diagnosis to date of screening, as reported by patients.
S.D.: Standard Deviation

TABLE 10
Diabetic foot ulcers duration at screening

| Average | S.D. | Minimum | Maximum |
|---|---|---|---|
| (months) | (months) | (months) | (months) |
| 10.2 | 14.7 | 0.0 | 72.5 |

Note:
N = 32.
Duration calculated from date of wound diagnosis to date of screening, as reported by patients.
S.D.: Standard Deviation.

TABLE 11
Venous leg ulcers duration at screening

| Average | S.D. | Minimum | Maximum |
|---|---|---|---|
| (months) | (months) | (months) | (months) |
| 424.9 | 93.9 | 0.0 | 518.0 |

Note:
N = 52.
Source: Duration calculated from date of wound diagnosis to date of screening, as reported by patients.
S.D.: Standard Deviation.

Further characteristics of the chronic wound type that each of the ninety-nine patients presented in the final analysis were suffering from included the area of skin and soft tissue that the given patient's wound encompassed and the bodily location of the wound. The size of the chronic ulcers was on average 10.96 cm² at the Screening visit, varying from 0.1 cm² up to 52.5 cm². The median size (all wounds combined) at study entry was 6.85 cm². (see Table 12 below).

TABLE 12
Chronic ulcers (all types) areas at screening and
First Treatment Visit

| Average | S.D. | Minimum | Maximum |
|---|---|---|---|
| (cm²) | (cm²) | (cm²) | (cm²) |
| 7.39 | 9.47 | 0.1 | 52.50 |

Note:
N = 99.
S.D.: Standard Deviation

The size of the chronic wound varied between the three chronic wound types, with DFUs presenting the smallest average size and VLUs presenting the largest average size at both the Screening and the First Treatment Visit time points. Wound size data for the PU patients is presented in Table 13, while that for the DFU patients is presented in Table 14, and that for the VLU patients is presented in Table 15.

TABLE 13
Pressure ulcers areas at Screening and
First Treatment Visit

| Average | S.D. | Minimum | Maximum |
|---|---|---|---|
| (cm²) | (cm²) | (cm²) | (cm²) |
| 4.29 | 5.36 | 0.1 | 21.30 |

Note:
N = 15.
S.D.: Standard Deviation

TABLE 14
Diabetic foot ulcers areas at Screening and First Treatment Visit

| Average | S.D. | Minimum | Maximum |
|---|---|---|---|
| (cm²) | (cm²) | (cm²) | (cm²) |
| 3.03 | 3.4 | 0.1 | 12.30 |

Note:
N = 32. S.D.: Standard Deviation

TABLE 15
Venous leg ulcers areas at Screening and First Treatment Visit

| Average | S.D. | Minimum | Maximum |
|---|---|---|---|
| (cm²) | (cm²) | (cm²) | (cm²) |
| 10.96 | 11.39 | 0.3 | 52.5 |

Note:
N = 52. S.D.: Standard Deviation

The bodily location of the chronic wounds in each of the three patient groups at the final analysis (ninety-nine patients) varied between patients in the given group. Tables 16, 17 and 18 present the data regarding the wound locations for the PU patients, the DFU patients and the VLU patients in the final analysis, respectively.

TABLE 16
Pressure ulcers location

| Pressure Ulcers/ | Total Number |
|---|---|
| Wound Location | of Wounds |
| Thigh | 1 |
| Sacrum/buttock | 9 |
| Heel | 5 |
| Total | 15 |

Note:
N = 15.

TABLE 17
Diabetic foot ulcers location

| Diabetic foot ulcers/ | Total Number |
|---|---|
| Wound Location | of Wounds |
| 1^{st} Dorsal Toe | 2 |
| 1^{st} Plantar Toe | 4 |
| 4^{th} Plantar Toe | 1 |
| Ankle | 5 |
| Arch of Foot | 2 |
| Ball of Foot | 13 |
| Dorsal Foot | 1 |
| Heel | 4 |
| Total | 32 |

Note:
N = 32.

TABLE 18
Venous leg ulcers location

| Venous leg ulcers/ | Total Number |
|---|---|
| Wound Location | of Wounds |
| External left ankle | 6 |
| External left leg | 2 |
| External right ankle | 7 |
| External right calf | 4 |
| External right leg | 3 |
| Internal left ankle | 12 |
| Internal left leg | 1 |
| Internal right ankle | 9 |
| Internal right calf | 3 |
| Internal right leg | 3 |
| External left calf | 1 |
| Internal left calf | 1 |
| Total | 52 |

Note:
N = 52.

All of the chronic ulcers treated in the study had failed on at least one form of treatment before. Overall, the vast majority had been previously treated with dressings and debridement, as per standard of care and clinical practice guidelines, with a complete list of prior treatments being presented in Tables 19 (all wounds), 20 (PU patients), 21 (DFU patients), and 22 (VLU patients). Dressings and medicated dressings were the most frequent treatments mentioned. Thirteen DFU patients had also already received systemic antibiotics, and fourteen VLU patients had a history of failed skin graft.

TABLE 19
Ulcers' prior treatments (all wounds combined)

| | Number |
|---|---|
| Description | of patients |
| Topical antibiotics | 7 |
| Compression (bands, socks) | 3 |
| Dressings (dry, wet, gels) | 26 |
| Medicated dressings | 102 |
| Systemic antibiotics | 6 |
| Topical disinfectants | 9 |
| (including topical ointments) | |
| Debridement | 14 |
| Grafting | 44 |
| Offloading | 1 |
| Collagen | 13 |
| Negative Pressure Wound Therapy | 3 |

Note:
N = 99. Patients might have more than one treatment.

TABLE 20
Pressure ulcers' prior treatments

| | Number |
|---|---|
| Description | of patients |
| Topical antibiotics | 2 |
| Dressings (dry, wet, gels) | 0 |
| Medicated dressings | 7 |
| Systemic antibiotics | 1 |
| Topical disinfectants | 2 |
| Topical Ointments | 3 |
| Debridement | 1 |
| Grafting | 0 |

Note:
N = 15. Patients might have more than one treatment

TABLE 21
Diabetic foot ulcers' prior treatments

| | Number |
|---|---|
| Description | of patients |
| Topical antibiotics | 4 |
| Compression (bands, socks) | 0 |
| Dressings (dry, wet, gels) | 5 |
| Medicated dressings | 20 |
| Systemic antibiotics | 4 |
| Topical disinfectants | 4 |
| Grafting | 1 |
| Offloading | 1 |

Note:
N = 32. Subjects might have more than one treatment.

TABLE 22
Venous leg ulcers' prior treatments

| | Number |
|---|---|
| Description | of patients |
| Topical antibiotics | 3 |
| Compression (bands, socks) | 3 |
| Dressings (dry, wet, gels) | 21 |
| Medicated dressings | 75 |
| Systemic antibiotics | 1 |
| Topical disinfectants | 1 |
| Topical Ointments | 2 |
| Debridement | 13 |
| Grafting | 13 |
| Collagen | 13 |
| Negative Pressure Wound Therapy | 3 |

Note:
N = 52.

Patient compliance to study visits from Screening to the end of study was excellent, which is not always observed as compliance may sometimes be an issue with patients affected by chronic wounds. Overall, 95.2% of study treatment visits planned by the protocol were received during the treatment period.

On average, patients were under investigational treatment for 80.45 days. The shortest period treatment was 0 days, for one of the DFU patients, whereas the longest treatment period was 224 days for four of the VLU patients. Table 23 presents the overall data for the ninety-nine patients' duration of investigational treatment.

TABLE 23
Investigational treatment duration

| Average | Standard | Minimum | Maximum |
|---|---|---|---|
| treatment | deviation | duration | duration |
| duration (days) | (days) | (days) | (days) |
| 80.45 | 51.18 | 0.0 | 224.0 |

Note:
N = 99.

The duration of investigational treatment varied depending on the chronic wound type group, and as shown in Table 24, the number of treatment days in the VLU group was lower than in the two other groups (PU and VLU). It might be explained by the high rate of wound closure in this group, and by the fact that these wounds responded overall quickly and favourably to the study treatment.

TABLE 24
Investigational treatment duration, by type of wound

| | Average | Standard | Minimum | Maximum |
|---|---|---|---|---|
| Type | treatment | deviation | duration | duration |
| of wound | duration (days) | (days) | (days) | (days) |
| PU | 87.53 | 37.14 | 19 | 142 |
| DFU | 70.28 | 66.6 | 0 | 224 |
| VLU | 84.67 | 43.13 | 3 | 173 |

Note:
N = 99. Average of all wounds shown in Table 23.

A majority of the thirty-three patients comprising the interim analysis of the study responded positively to the treatment with the biophotonic composition; a total of twenty-one of the patients were considered to be full responders, wherein a full responder was defined as having a decrease of the wound size area of more than 90% at the end of the study period and/or decrease of more than 50% of the size in 15 days or less. This cohort of full responders comprised twelve DFU patients, eight VLU patients and one PU patient. Furthermore, three patients in each of DFU and VLU wound types were considered to be partial responders, wherein a partial responder was defined as having a decrease of the size of the wound during the study period, but without meeting the criteria of full responder. Nine of the full responder DFU patients experienced a total closure of their wounds by the end of their participation in the study, while seven of the eight VLU experienced full closure by the end of their participation (with the eighth full responder VLU experiencing a 97% decrease in wound area by this patients end of participation). The full responder PU patient experienced a complete wound closure by the day of their last treatment visit (day 47). Overall, of the thirty-three patients presented in the interim analysis, 16 patients (48.5% of the total number of patients) experienced a full closure of their wound totally during the study period, and the mean time to reach total closure was 46.8 days. This mean time varied depending on the type of wound; it was lower for DFUs (mean time of 37.2 days), whereas it was higher for PUs (47.0 days) and VLUs (53.6 days). Of the three partial responders in the VLU cohort, two of these patients' wounds were graft-ready by their completion of the study, and for the three partial responders in the DFU patient cohort, one of these patients' wound was graft ready by the end of the patient's participation in the study.

In the ninety-nine patient study, the average number of treatments by types of wounds are (i) 23.90 treatments for patients with VLU, (ii) 18.31 treatments for patients with DFU, and (iii) 23.40 treatments for patients with PU. Significant variations of the wound size area as compared to baseline were found for VLU (p<0.001) and DFU (p=0.001). In the study, 47 wounds closed completely during the study period, especially VLUs (26) and DFUs (16). It represents 47.5% of the wounds treated during the study period. By type of wounds, the results are (i) 50% of the VLU wounds closed completely; (ii) 50% of the DFU wounds closed completely; and (iii) 33.3% of the PU wounds closed completely.

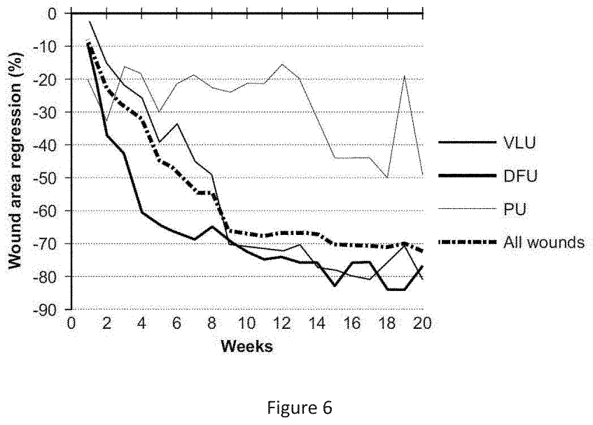

Looking at the trajectories of relative wound area regression over time, superior results were obtained with VLUs and DFUs, showing similar results. As shown in FIG. 6, the mean time to reach a regression of 50% of the wound area was approximately 8 weeks for VLUs and 3.5 weeks for DFUs.

### Incidence of Wound Breakdown:

To confirm the complete closure of a wound, the absence of breakdown in the two weeks following its closure is confirmed.

Incidence of wound breakdown was assessed at two timepoints during the follow-up period:
after the two-week evaluation period following wound closure: two wounds (5.13%) had a wound breakdown (N=39);
after the eight-week evaluation period following wound closure: two wounds (5.71%) had a wound breakdown (N=35).

These low percentages of wound breakdown show that, when a chronic wound totally closed following treatment, 95% of them remain totally closed after the two-week follow-up period, and even after the eight-week follow-up period.

This low incidence of wound breakdown at two weeks and eight weeks' post-closure confirms the efficacy profile and long-lasting action of a biophotonic composition of the present disclosure on wound closure.

### Wounds Ready to Skin Graft According to Clinicians' Opinion:

The results in terms of wound bed preparation were also positive in this study. Investigators were asked to assess regularly during the study period if the wounds became ready to skin graft. Even if the wounds had a profile of hard to heal non responsive wounds at study entry, with a high rate of prognosis factors of poor healing, more than two thirds of the VLU (69.2%) and DFU (68.8%) wounds became ready to skin graft at one point during the study period.

### Effects of Treatment on Pain:

Specific information on pain that the patients were experiencing in relation to their particular chronic wound type was collected in an objective fashion through the completion of an “Ulcer assessment” questionnaire by the clinical investigators at each Treatment visit. In this questionnaire, investigators were asked to assess the pain level of their patients throughout the study.

Table 25 summarizes the number of patients in the Interim Study with pain at Baseline according to the investigators, by type of wound. Baseline is defined as the first treatment visit, before any study treatment. As can be seen from Table 25, fourteen days after the treatment with the biophotonic composition, 100% of the patients who had pain on the treated wound at baseline had no more pain.

TABLE 25
Number of patients with pain reported by
investigators in Interim Study—summary by type of wound

| | Number of | Number of | Difference |
|---|---|---|---|
| | patients | patients declaring | between Baseline |
| Type | declaring pain | pain 14 days | and Day 14 |
| of wound | at baseline | after baseline | post Baseline |
| PU | 0 | 0 | 0 |
| DFU | 2 | 0 | −2 (−100%) |
| VLU | 7 | 0 | −7 (−100%) |
| Total | 9 | 0 | −9 (100%) |

Note:
N = 33.

Table 26 provides the number of Interim Study patients reporting pain over time for all wounds. The results show that even if the pain level was generally not very frequent at time of first treatment (nine patients at Baseline), it disappeared systematically during the treatment period, regardless of the pain intensity at baseline and the type of wound.

TABLE 26
Number of patients with pain reported by
investigators in Interim Study—time to no pain—all wounds

| | |
|---|---|
| Number of patients declaring pain at baseline | 9 |
| Minimum time to no-pain (Days) | 7 |
| Maximum time to no-pain (Days) | 9 |
| Average time to no-pain (days) | 9.78 |

Note:
N = 33.

Despite the fact that procedures such as negative pressure therapy for treatment of chronic ulcers, especially venous leg ulcers, may generally be considered as painful and/or uncomfortable for patients who have the ability to feel pain, average time to no pain in patients receiving the biophotonic treatment in the Interim Study was 9.78 days, meaning that a maximum of 3 treatments with the biophotonic composition was needed to remove that pain. Once gone, the pain never reappeared again during the study period.

The data presented in Tables 27 and 28 show an average time to no pain of 1.67 days for DFUs and 11.86 days to no pain for VLUs, respectively. No patient with PU reported pain.

TABLE 27
Number of patients with pain reported by
investigators in Interim Study—time to no pain—DFU

| | |
|---|---|
| Number of patients declaring pain at baseline | 2 |
| Minimum time to no-pain (Days) | 1 |
| Maximum time to no-pain (Days) | 4 |
| Average time to no-pain (days) | 1.67 |

Note:
N = 17.

TABLE 28
Pain levels reported by investigators
in Interim Study—time to no pain—VLU

| | |
|---|---|
| Number of patients declaring pain at baseline | 7 |
| Minimum time to no-pain (Days) | 7 |
| Maximum time to no-pain (Days) | 14 |
| Average time to no-pain (days) | 11.86 |

Note:
N = 13.

It is to be noted that the clinical investigators were required to declare any apparition of pain not present at baseline as an adverse event. However, no adverse event on pain was declared throughout the study period for the thirty-three patients presented in the interim analysis, indicating that no pain linked to the procedure was reported by investigators during the study period.

In summary, the results showed that if pain was initially present before the first treatment with the biophotonic composition, it disappeared systematically and quickly once the first treatment was initiated.

### Local Clinical Signs of Wound Colonization Over Time:

Investigators were asked at the first Treatment visit to assess different aspects of the wound and peri-wound skin, including the presence of local signs of colonization or infection (Redness, Pain, Heat, Pus and Swelling);

General characteristics at first treatment visit were:
As expected with these hard to heal chronic wounds, the majority of them had local clinical signs of bacterial colonization at study entry, with redness (37.4% all wounds combined) and heat (7.1%). It concerned especially VLU, with 57.7% having redness and 11.5% having heat. There was no clinical sign of bacterial colonization for DFUs and PUs;
This hypothesis of a probable colonization of some of the wounds at study entry is reinforced by the amount of exudate: 26.9% of high exudate level for VLUs and 20.0% for DFUs (18.2% overall);
The great majority (98.0%) had no visible presence of pus.

A fast regression of these clinical signs of bacterial colonization was observed as soon as Week 4:
Redness: from 58% to 36%;
Pain: from 52% to 16%;
Swelling: from 29% to 14%;
No more wound with pus.
   Complete results of these different assessments at the first treatment visit are presented in the table below.

TABLE 29
Local signs of colonization/Infection by wound type at 1^{st} Treatment
Visit

| | | Wound type | | | | | | | |
|---|---|---|---|---|---|---|---|---|---|
| | | VLU | | DFU | | PU | | Total | |
| | | N | % | N | % | N | % | N | % |
| Red- | YES | 30 | 57.7% | 6 | 18.8% | 1 | 6.7% | 37 | 37.4% |
| ness | NO | 22 | 42.3% | 26 | 81.3% | 14 | 93.3% | 62 | 62.6% |
| | Total | 52 | 100.0% | 32 | 100.0% | 15 | 100.0% | 99 | 100.0% |
| Pain | YES | 27 | 51.9% | 1 | 3.1% | 0 | 0.0% | 28 | 28.3% |
| | NO | 25 | 48.1% | 31 | 96.9% | 15 | 100.0% | 71 | 71.7% |
| | Total | 52 | 100.0% | 32 | 100.0% | 15 | 100.0% | 99 | 100.0% |
| Heat | YES | 6 | 11.5% | 1 | 3.1% | 0 | .0% | 7 | 7.1% |
| | NO | 46 | 88.5% | 31 | 96.9% | 15 | 100.0% | 92 | 92.9% |
| | Total | 52 | 100.0% | 32 | 100.0% | 15 | 100.0% | 99 | 100.0% |
| Pus | YES | 1 | 1.9% | 1 | 3.1% | 0 | .0% | 2 | 2.0% |
| | NO | 51 | 98.1% | 31 | 96.9% | 15 | 100.0% | 97 | 98.0% |
| | Total | 52 | 100.0% | 32 | 100.0% | 15 | 100.0% | 99 | 100.0% |
| Swell- | YES | 15 | 28.8% | 3 | 9.4% | 3 | 20.0% | 21 | 21.2% |
| ing | NO | 37 | 71.2% | 29 | 90.6% | 12 | 80.0% | 78 | 78.8% |
| | Total | 52 | 100.0% | 32 | 100.0% | 15 | 100.0% | 99 | 100.0% |

Note:
N = 99.

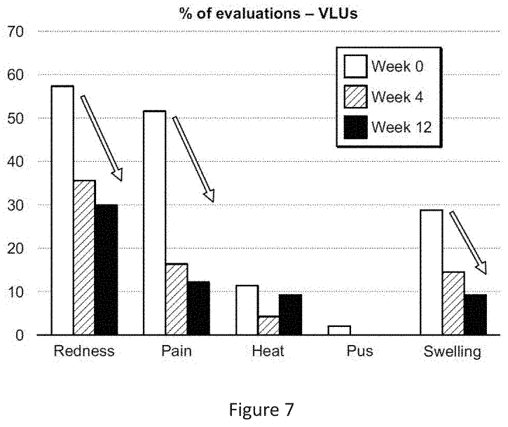

These data and their evolution from first Treatment visit to Week 4 and Week 12 are also summarized in FIG. 7. It is interesting to note a specific and fast action on pain reduction, when pain was present at the first study visit (mainly for VLUs).

The number of patients declaring pain at baseline (9 out of 33) in the Interim Study is to be relativized as PU and DFU patients do not usually suffer from pain due to various neurological co-morbidities. Considering only the VLU population, which is the population of patients mostly concerned by pain among chronic ulcers, pain was present at baseline in seven patients out of 13, indicating that 53.8% of this patient population was experiencing pain prior to receiving the first treatment with the biophotonic composition. Pain systematically disappeared once the treatment with biophotonic composition was initiated, with an average time to no-pain of only 11.9 days (indicating that a maximum of four treatments with the biophotonic composition for an elimination of pain), with a maximum time being 14 days. Once gone, the pain never reappeared and no adverse event concerning apparition or exacerbation of pain occurred during the study.

Patients were also assessed from a “quality of life” perspective in relation to their having received the course of treatment with the biophotonic composition. To evaluate such a change, an Italian version of the Cardiff Wound Impact Schedule (CWIS), a validated quality of life questionnaire specifically designed for patients suffering from chronic ulcers, was provided to the patients during the study period. The CWIS includes a total score and three main components (“sub-scores”): “Social life”, “Well-being” and “Physical symptoms and daily living”, with the questionnaire being designed to be self-administered and completed on an individual basis. The questionnaire was to be completed by patients on two occasions: at the Screening visit and at the first Follow-up visit.

Based on the validated Scoring instructions, if the patient responded by providing a higher score in comparison to their score at Screening, the quality of life was assessed as better. In other words, an increase of the score between the Screening visit and Follow-up 1 Visit indicated an increase of the overall quality of life of the patient (for the total score) or of one of its components. An increase of the sub-score indicated an improvement of the particular quality of life component for the patient.

For the thirty-three patients comprising the interim analysis, a large increase (26.4%, based on a total score of 156.6 at Screening versus a total score of 248.5 at the first Follow-up visit) in the total score (all wound types combined) resulted during the course of these patients participation in study period; the increase in the overall total score confirmed a positive impact of the study treatment on overall aspects of quality of life for patients with chronic wounds, and was considered to be reinforced by a positive impact on the reduction of pain experienced by a large number of the treated patients. This positive tendency with respect to the patients' quality of life scores was also observed with the three sub-scores: Social life increased by 22.3% (score of 73 at Screening versus a score of 89.3 at the first Follow-up visit), Well-being increased by 44.7% (score of 49 at Screening versus a score of 70.9 at the first Follow-up visit) and Physical symptoms and daily living increased by 18.4% (score of 74.9 at Screening versus a score of 88.3 at the first Follow-up visit).

For the ninety-nine patients in the study, the total score increased during the study period for all wounds (+15.4%), confirming the positive impact of the study treatment on overall aspects of quality of life for subjects with chronic wounds. This positive tendency was also observed with the three sub-scores: Social life (+11.1%), Well-being (+27.8%) and Physical symptoms/Daily living (+11.3%). These results confirm the high level of patients' confidence in the efficacy of the treatment throughout the study period.

### Example 2—Chronic Wound

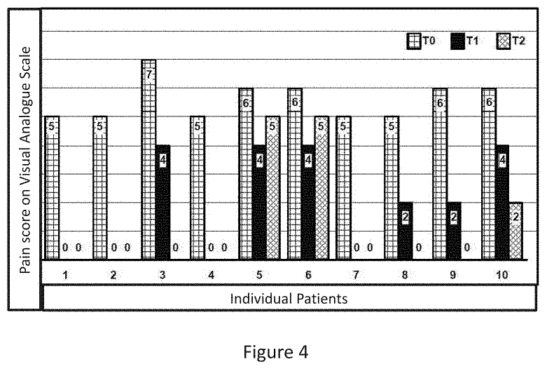

A clinical study was performed involving ten VLU patients who received treatment of the biophotonic composition described in Example 1. Patients were treated twice per week over the course of a 16-week treatment period, with the biophotonic composition being applied, illuminated with the multi-LED light, and thereafter removed from the treated wound in a manner as described in Example 1. The treated patients themselves provided a subjective evaluation of their wound associated pain using a 100 millimeter Visual Analog Scale (VAS). Patients provided their VAS pain evaluations at T0 (upon receiving the first treatment with the biophotonic composition), at T1 (upon receiving their second treatment during the first week) and at T2 (upon receiving the third treatment with biophotonic composition, i.e. the first treatment of the second week). The results with respect to the patients VAS assessment of their wound associated pain are presented in FIG. 4. The results indicated that for all ten patients experienced a reduction in their pain scores by the time of having received three treatments with the biophotonic composition (at T2), with seven of the ten patients reporting a complete absence of pain by the T2 point.

### Example 3—Acute Wound

A study was performed with 42 patients having bilateral breast reduction.

Comparison A: Patients were randomized to Comparison A and were re-randomized to get a biophotonic treatment using a biophotonic composition as described herein (Eosin Y at 0.305 mg/ml, 12% carbamide peroxide) either once weekly (Group A1) or twice weekly (Group A2) starting on Day 7 post-surgery (breast reduction). The breast wound receiving the biophotonic treatment was randomly selected. The breast wound receiving Silicone Sheets (as described below) was also randomly selected and received a first application of Silicone Sheets on Day 21 post-surgery. The biophotonic treatment was applied for a minimum period of 6 weeks, up to a maximum period of 8 weeks. The Silicone Sheets were applied for a minimum period of 8 weeks and up to a maximum period of 12 weeks.

Comparison B: Patients were randomized to Comparison B and were re-randomized to get a biophotonic treatment using a biophotonic composition as described herein (Eosin Y at 0.305 mg/ml, 12% carbamide peroxide) either applied once weekly (Group B1) or twice weekly (Group B2) starting on Day 21 post-surgery (breast reduction). The breast wound receiving the biophotonic treatment was randomly selected. The breast wound receiving Silicone Sheets was also randomly selected and received a first application of Silicone Sheets on Day 21 post-surgery. The biophotonic treatment was applied for a minimum period of 6 weeks, up to a maximum period of 8 weeks. The Silicone Sheets were applied for a minimum period of 8 weeks and up to a maximum period of 12 weeks.

Comparison C: Patients were randomized to Comparison C and were re-randomized to get double (two consecutive treatments) the biophotonic treatment using a biophotonic composition as described herein (Eosin Y at 0.305 mg/ml, 12% carbamide peroxide) which was applied once weekly starting either at Day 7 (Group C1) or at Day 21 (Group C2) post-surgery (breast reduction). The breast wound receiving the biophotonic treatment was randomly selected. The breast wound receiving Silicone Sheets was also randomly selected and received a first application of Silicone Sheets on Day 21 post-surgery (breast reduction). The double biophotonic treatment was applied for a minimum period of 6 weeks, up to a maximum period of 8 weeks. The Silicone Sheets were applied for a minimum period of 8 weeks and up to a maximum period of 12 weeks.

Biophotonic treatment: An amount of the biophotonic composition was topically applied onto the painful area of the breast having the acute wound and the biophotonic composition was illuminated for a treatment period of 5 minutes using a phototherapeutic lamp. Following the 5 minute illumination period, the biophotonic composition was removed (e.g., washed off) from the skin. For patients receiving the double biophotonic treatment (two consecutive biophotonic treatments (Comparison C)), once the first illumination of the area of pain associated with the acute wound was performed, the used biophotonic composition was removed and a second amount of the biophotonic composition was applied right away onto the same treatment area. The second application of biophotonic composition was then illuminated for 5 minutes using the phototherapeutic lamp. The Silicone Sheets were used and applied as per manufacturers' instructions.

Phototherapeutic lamp: The study was performed using a phototherapeutic device delivering non-coherent blue light with peak wavelengths in the range of 440-460 nm at a distance of 5 cm (distance between the light source and the surface to the illuminated).

Silicone sheets (CICA-CARE® SILICONE SHEETING): CICA-CARE® SILICONE SHEETING is a self-adhesive silicone gel sheet medically proven to be up to 90% effective in the improvement of red, dark or raised scars. It is designed for use in the management of both existing and new hypertrophic and keloid scars (red and raised) and as a preventive therapy on closed wounds to prevent hypertrophic and keloid scars (red and raised).

Biophotonic Treatment Period: Patients could be randomized to one of three different comparisons:
Comparison A: Application of the biophotonic composition initiated on Day 7 post-surgery, once or twice weekly for a period of 6-8 weeks, vs. Silicone Sheets initiated on Day 21 post-surgery for a period of 8-12 weeks;
Comparison B: Application of the biophotonic composition initiated on Day 21 post-surgery, once or twice weekly for a period of 6-8 weeks, vs. Silicone Sheets initiated on Day 21 post-surgery for a period of 8-12 weeks;
Comparison C: Application of the biophotonic composition initiated on Day 7 or on Day 21 post-surgery, once weekly for a period of 6-8 weeks, vs. Silicone Sheets initiated on Day 21 post-surgery for a period of 8-12 weeks.

Scarring Criteria Assessment For the study of Example 3, the Patient and Observer Scar Assessment Scale (POSAS), designed to be used by both the clinician and the patient (Draaijers et al., 2004 “The patient and observer scar assessment scale: a reliable and feasible tool for scar evaluation. Plast Reconstr Surg; vol. 113: pages 1960-1965); van de Kar et al., 2005 “Reliable and feasible evaluation of linear scars by the Patient and Observer Scar Assessment Scale” Plast Reconstr Surg, vol. 116, pages 514-522), was utilized to evaluate various scarring criteria subsequent to the breast reduction surgery and in light of the patient receiving post-surgery either a biophotonic treatment regimen or application of Silicone Sheets. The clinician component of the POSAS assessment comprised an evaluation of the scar looking at vascularity, pigmentation, thickness, relief, pliability and importance of surface area, whereas the patient component of the POSAS comprised an assessment of pain, itching, color, stiffness, thickness, contour irregularities and overall opinion.

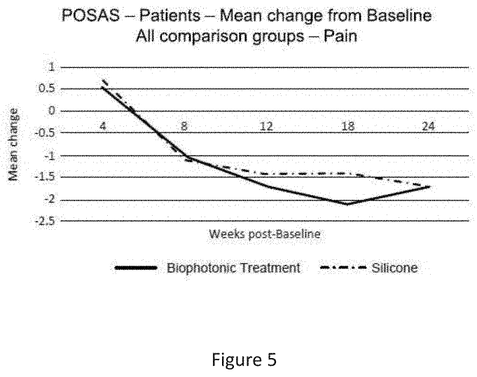

Results Regarding the Pain Criteria Evaluation—Reduction of Pain FIG. 5 presents data showing a mean change from baseline, with respect to a reduction of pain experienced by the patients receiving the biophotonic treatment (BT) in comparison those receiving the Silicone Sheets treatment following their breast reduction surgery. As shown in FIG. 5, patients receiving the biophotonic treatment experienced a substantial reduction in their pain assessment in the 4 to 8 week post-baseline period, with the reduction being at least equivalent to that experienced by the patients receiving the Silicone sheet treatment (the latter considered to be the “standard of care”). Subsequent to the 8 week post-baseline time point, however, patients receiving the biophotonic treatment continued to experience a further reduction in their amount of pain in comparison to a relative levelling-off of the reduction of pain experienced subsequent to the 8 week post-baseline point in those patients receiving the Silicone sheet treatment.

### INCORPORATION BY REFERENCE

All references cited in this specification, and their references, are incorporated by reference herein in their entirety where appropriate for teachings of additional or alternative details, features, and/or technical background.

### EQUIVALENTS

While the disclosure has been particularly shown and described with reference to particular embodiments, it will be appreciated that variations of the above-disclosed and other features and functions, or alternatives thereof, may be desirably combined into many other different systems or applications. Also, that various presently unforeseen or unanticipated alternatives, modifications, variations or improvements therein may be subsequently made by those skilled in the art which are also intended to be encompassed by the following embodiments.

1. A method for reducing pain associated with a medical condition in a subject, comprising:
a) topically applying a composition comprising at least one photoactivator and a pharmaceutically acceptable carrier; and
b) exposing said composition to actinic light to cause activation of the composition, wherein the pain associated with the medical condition is treated non-systemically.

2. The method according to claim 1, wherein the pain does not originate from a stimulus in an orofacial region.

3. The method according to claim 1, wherein the reduction of pain is not dependent on the treatment of the underlying medical condition.

4. The method according claim 1, wherein the composition is a biophotonic composition.

5. The method according claim 1, wherein the photoactivator is selected from the group consisting of a xanthene derivative dye, an azo dye, a biological stain, and a carotenoid.

6. The method according to claim 5, wherein the at least one photoactivator is selected from the group consisting of eosin, erythrosine, and Saffron red powder.

7. (canceled)

8. The method according to claim 6, wherein the at least one photoactivator is eosin Y.

9. The method according to claim 6, wherein the at least one photoactivator is erythrosine B.

10. The method according to claim 1, wherein the at least one photoactivator is present in an amount of at least about 0.02% by weight of the composition.

11.-34. (canceled)

35. The method according to claim 1, wherein the composition further comprises at least one healing factor.

36. The method according to claim 35, wherein the healing factor is selected from the group consisting of hyaluronic acid, glucosamine, and allantoin.

37.-51. (canceled)

52. The method according to claim 35, wherein the composition further comprises at least one salt selected from the group consisting of:
one or more bicarbonate salts selected from the group consisting of ammonium bicarbonate, caesium bicarbonate, potassium bicarbonate, sodium bicarbonate, choline bicarbonate, aminoguanidine bicarbonate, tetraethyl ammonium bicarbonate, sodium bicarbonate, and potassium bicarbonate;
one or more carbonate salts selected from the group consisting of barium carbonate, beryllium carbonate, caesium carbonate, calcium carbonate, cobalt (II) carbonate, copper (II) carbonate, lithium carbonate, magnesium carbonate, nickel (II) carbonate, potassium carbonate, sodium carbonate, zinc carbonate, sodium carbonate, calcium carbonate, and potassium bicarbonate; and
a combination of the foregoing salts.

53.-56. (canceled)

57. The method according to claim 1, wherein the pain is acute or chronic pain.

58. The method according to claim 57, wherein the chronic pain is nociceptive pain or neuropathic pain.

59. The method according to claim 1, wherein the pain is selected from the group consisting of widespread pain, localized pain, nociceptive pain, inflammatory pain, peripheral neuropathic pain, peripheral neurogenic pain, peripheral neuralgia, low back pain, postoperative pain, visceral pain, and pelvic pain; allodynia; anesthesia dolorosa; causalgia; dysesthesia; fibromyalgia; hyperalgesia; hyperesthesia; ischemic pain; sciatic pain; pain associated with cystitis including, but not limited to, interstitial cystitis; pain associated with multiple sclerosis; pain associated with arthritis; pain associated with osteoarthritis; pain associated with rheumatoid arthritis; pain associated with chronic wounds; pain associated with burns; and pain associated with cancer.

60.-65. (canceled)

66. The method according to claim 1, wherein the pain is associated with postoperative care.

67. The method according to claim 1, wherein said composition is exposed to actinic light for at least one treatment period of from about 1 second to about 9 minutes per cm² of an area to be treated, the actinic light having a wavelength between about 400 nm and about 700 nm, the illumination thereof being in continuous motion over the area to be treated.

68.-74. (canceled)

75. The method according to claim 1, wherein said composition is exposed to at least two treatment periods of actinic light wherein each treatment period is from about 1 minute to about 5 minutes per cm of an area to be treated, and wherein each treatment period is followed by a resting interval of from about 1 minute to about 5 minutes.

76. (canceled)

77. The method according claim 1, further comprising:
a) topically applying the composition to the subject's area of pain;
b) exposing the subject's area of pain to actinic light for a treatment period of from about 1 minute to about 10 minutes;
c) removing the source of actinic light away from the subject's area of pain treated for a resting interval of from about 1 minute to about 5 minutes;
d) exposing the subject's area of pain to actinic light for a second treatment period of from about 1 minute to about 10 minutes; and
wherein the first exposure to actinic light activates the composition.

78.-91. (canceled)

92. A method of reducing pain associated with a medical condition in a subject, comprising:
a) identifying area of the pain;
b) topically applying on the subject's areas of pain a composition comprising at least one photoactivator and a pharmaceutically acceptable carrier; and
c) exposing the subject's area of pain to actinic light for a time sufficient to activate said composition.

93.-94. (canceled)
